(19) United States
(12) Patent Application Publication
(10) Pub. No.: US 2003/0022835 A1
(43) Pub. Date: Jan. 30, 2003

(54) Compositions isolated from skin cells and methods for their use

(75) Inventors: James D. Watson, Auckland (NZ); Lorna Strachan, Auckland (NZ); Matthew Sleeman, Weston Colville (GB); Rene Onrust, Mercer Is, WA (US); James G. Murison, Auckland (NZ); Krishanand D. Kumble, Los Altos, CA (US)

Correspondence Address: SPECKMAN LAW GROUP, 1501 WESTERN AVE, SUITE 100, SEATTLE, WA, 98101, US

(73) Assignee: Genesis Research and Development Corporation Limited, Auckland (NZ)

(21) Appl. No.: 10/152,661

(22) Filed: May 20, 2002

Related U.S. Application Data
(63) Continuation-in-part of application No. 09/866,050, filed on May 24, 2001.
Application No. 09/866,050 is a continuation-in-part of application No. 09/312,283, filed on May 14, 1999.
Application No. 09/312,283 is a continuation-in-part of application No. 09/188,930, filed on Nov. 9, 1998, now Pat. No. 6,150,502.
Application No. 09/188,930 is a continuation-in-part of application No. 09/069,726, filed on Apr. 29, 1998, now abandoned.
(60) Provisional application No. 60/206,650, filed on May 24, 2000.
(60) Provisional application No. 60/221,232, filed on Jul. 25, 2000.

(30) Foreign Application Priority Data
Apr. 29, 1999 (WO) PCT/NZ99/00051

(51) Int. Cl. A61K 38/17; C12P 21/02; C12N 5/06; C07H 21/04; C07K 14/435

(52) U.S. Cl. 514/12; 530/350; 536/23.5; 435/69.1; 435/320.1; 435/325

(57) ABSTRACT

Isolated polynucleotides encoding polypeptides expressed in mammalian skin cells are provided, together with expression vectors and host cells comprising such isolated polynucleotides. Methods for the use of such polynucleotides and polypeptides are also provided.

### REFERENCE TO RELATED APPLICATIONS

This application is a continuation-in-part of U.S. patent application Ser. No. 09/866,050, filed May 24, 2001, which is a continuation-in-part of U.S. application Ser. No. 09/312,283, filed May 14, 1999, which is a continuation-in-part of U.S. application Ser. No. 09/188,930, filed Nov. 9, 1998, now U.S. Pat. No. 6,150,502, which is a continuation-in-part of U.S. application Ser. No. 09/069,726, filed Apr. 29, 1998, now abandoned, and claims priority to International Patent Application No. PCT/NZ99/00051, filed Apr. 29, 1999, U.S. Provisional Application No. 60/206,650, filed May 24, 2000, and U.S. Provisional Application No. 60/221,232, filed Jul. 25, 2000.

### TECHNICAL FIELD OF THE INVENTION

This invention relates to polynucleotides and polypeptides expressed in skin cells, and various methods for treating a patient involving administration of a polypeptide or polynucleotide of the present invention.

### REFERENCE TO SEQUENCE LISTING SUBMITTED ON COMPACT DISC

This application incorporates by reference in its entirety the Sequence Listing contained in the accompanying two compact discs, one of which is a duplicate copy. Each CD contains the following file: 1011C5 SEQLIST.txt, having a date of creation of May 17, 2002.

### BACKGROUND OF THE INVENTION

The skin is the largest organ in the body and serves as a protective cover. The loss of skin, as occurs in a badly burned person, may lead to death owing to the absence of a barrier against infection by external microbial organisms, as well as loss of body temperature and body fluids.

Skin tissue is composed of several layers. The outermost layer is the epidermis which is supported by a basement membrane and overlies the dermis. Beneath the dermis is loose connective tissue and fascia which cover muscles or bony tissue. The skin is a self-renewing tissue in that cells are constantly being formed and shed. The deepest cells of the epidermis are the basal cells, which are enriched in cells capable of replication. Such replicating cells are called progenitor or stem cells. Replicating cells in turn give rise to daughter cells called ‘transit amplifying cells’. These cells undergo differentiation and maturation into keratinocytes (mature skin cells) as they move from the basal layer to the more superficial layers of the epidermis. In the process, keratinocytes become cornified and are ultimately shed from the skin surface. Other cells in the epidermis include melanocytes which synthesize melanin, the pigment responsible for protection against sunlight. The Langerhans cell also resides in the epidermis and functions as a cell which processes foreign proteins for presentation to the immune system.

The dermis contains nerves, blood and lymphatic vessels, fibrous and fatty tissue. Within the dermis are fibroblasts, macrophages and mast cells. Both the epidermis and dermis are penetrated by sweat, or sebaceous glands and hair follicles. Each strand of hair is derived from a hair follicle. When hair is plucked out, the hair re-grows from epithelial cells directed by the dermal papillae of the hair follicle.

When the skin surface is breached, for example in a wound, the stem cells proliferate and daughter keratinocytes migrate across the wound to reseal the tissues. The skin cells therefore possess genes activated in response to trauma. The products of these genes include several growth factors, such as epidermal growth factor, which mediate the proliferation of skin cells. The genes that are activated in the skin, and the protein products of such genes, may be developed as agents for the treatment of skin wounds. Additional growth factors derived from skin cells may also influence growth of other cell types. As skin cancers are a disorder of the growth of skin cells, proteins derived from skin that regulate cellular growth may be developed as agents for the treatment of skin cancers. Skin derived proteins that regulate the production of melanin may be useful as agents, which protect skin against unwanted effects of sunlight.

Keratinocytes are known to secrete cytokines and express various cell surface proteins. Cytokines and cell surface molecules are proteins, which play an important role in the inflammatory response against infection, and also in autoimmune diseases affecting the skin. Genes and their protein products that are expressed by skin cells may thus be developed into agents for the treatment of inflammatory disorders affecting the skin.

Hair is an important part of a person's individuality. Disorders of the skin may lead to hair loss. Alopecia areata is a disease characterized by the patchy loss of hair over the scalp. Total baldness is a side effect of drug treatment for cancer. The growth and development of hair is mediated by the effects of genes expressed in skin and dermal papillae. Such genes and their protein products may be usefully developed into agents for the treatment of disorders of the hair follicle.

New treatments are required to hasten the healing of skin wounds, to prevent the loss of hair, enhance the re-growth of hair or removal of hair, and to treat autoimmune and inflammatory skin diseases more effectively and without adverse effects. More effective treatments of skin cancers are also required. There thus remains a need in the art for the identification and isolation of genes encoding proteins expressed in the skin, for use in the development of therapeutic agents for the treatment of disorders including those associated with skin.

### SUMMARY OF THE INVENTION

The present invention provides polypeptides and functional portions of polypeptides, which may be expressed in skin cells, together with polynucleotides encoding such polypeptides or functional portions thereof, expression vectors and host cells comprising such polynucleotides, and methods for their use.

In specific embodiments, isolated polynucleotides are provided that comprise a polynucleotide selected from the group consisting of: (a) sequences recited in SEQ ID NOS: 1-119, 198-276, 349-372, 399-405, 410-412, 416, 418-455, 464, 466-487, 510, 511 and 514-623; (b) complements of the sequences recited in SEQ ID NOS: 1-119, 198-276, 349-372, 399-405, 410-412, 416, 418-455, 464, 466-487, 510, 511 and 514-623; (c) reverse complements of the sequences recited in SEQ ID NOS: 1-119, 198-276, 349-372, 399-405, 410-412, 416, 418-455, 464, 466-487, 510, 511 and 514-623; (d) reverse sequences of the sequences recited in SEQ ID NOS: 1-119, 198-276, 349-372, 399-405, 410-412, 416, 418-455, 464, 466-487, 510, 511 and 514-623; (e) sequences having a 99% probability of being the same as a sequence of (a)-(d); and (f) sequences having at least 75%, 90% or 95% identity to a sequence of (a)-(d).

In further embodiments, the present invention provides isolated polypeptides comprising an amino acid sequence selected from the group consisting of: (a) sequences provided in SEQ ID NOS: 120-197, 275-348, 373-398, 406-409, 413-415, 417, 456-463, 465, 488-509, 512, 513 and 624-725; and (b) sequences having at least 75%, 90% or 95% identity to a sequence provided in SEQ ID NOS: 120-197, 275-348, 373-398, 406-409, 413-415, 417, 456-463, 465, 488-509, 512, 513 and 624-725, together with isolated polynucleotides encoding such polypeptides. Isolated polypeptides which comprise at least a functional portion of an amino acid sequence selected from the group consisting of: (a) sequences provided in SEQ ID NOS: 120-197, 275-348, 373-398, 406-409, 413-415, 417, 456-463, 465, 488-509, 512, 513 and 624-725, and variants thereof, are also provided.

In another aspect, the present invention provides fusion proteins comprising at least one polypeptide of the present invention.

In related embodiments, the present invention provides expression vectors comprising the above polynucleotides, together with host cells transformed with such vectors.

As detailed below, the isolated polynucleotides and polypeptides of the present invention may be usefully employed in the preparation of therapeutic agents for the treatment of disorders such as skin wounds, cancers, growth and developmental defects, and inflammatory diseases. The present invention thus further provides compositions comprising an inventive polypeptide, fusion protein, or polynucleotide encoding such a polypeptide, together with at least one component selected from the group consisting of: physiologically acceptable carriers and immunostimulants.

In further aspects, the present invention provides methods for the treatment of a disorder in a subject, together with methods for modulating skin inflammation, stimulating epithelial cell growth, stimulating keratinocyte growth and motility, inhibiting the growth of epithelial-derived cancer cells, inhibiting angiogenesis and vascularization of tumors, modulating the growth of blood vessels, inhibiting the binding of HIV-1 to leukocytes, treatment of an inflammatory disease or for the treatment of cancer in a subject in a subject, comprising administering to the subject a composition described herein.

In yet a further aspect, methods for the treatment of a disorder by reducing the effective amount, inactivating, and/or inhibiting the activity of an inventive polypeptide or a polynucleotide that encodes such a polypeptide are provided.

The above-mentioned and additional features of the present invention, together with the manner of obtaining them, will be best understood by reference to the following more detailed description. All references disclosed herein are incorporated herein by reference in their entirety as if each was incorporated individually.

### BRIEF DESCRIPTION OF THE DRAWINGS

FIG. 1 shows the results of a Northern analysis of the distribution of huTR1 mRNA in human tissues. Key: He, Heart; Br, Brain; Pl, Placenta; Lu, Lung; Li, Liver; SM, Skeletal muscle; Ki, Kidney; Sp, Spleen; Th, Thymus; Pr, Prostate; Ov, Ovary.

FIG. 2 shows the results of a MAP kinase assay of muTR1a and huTR1a. MuTR1a (500 ng/ml), huTR1a (100 ng/ml) or LPS (3 pg/ml) were added as described in the text.

FIG. 3 shows the stimulation of growth of neonatal foreskin keratinocytes by muTR1a.

FIG. 4 shows the stimulation of growth of the transformed human keratinocyte cell line HaCaT by muTR1a and huTR1a.

FIG. 5 shows the inhibition of growth of the human epidermal carcinoma cell line A431 by muTR1 a and huTR1a.

FIG. 6 shows the inhibition of IL-2 induced growth of concanavalin A-stimulated murine splenocytes by KS2a.

FIG. 7 shows the stimulation of growth of rat intestinal epithelial cells (IEC-18) by a combination of KS3a plus apo-transferrin.

FIG. 8 illustrates the oxidative burst effect of TR-1 (100 ng/ml), muKS1 (100 ng/ml), SDF1α (100 ng/ml), and fMLP (10 μM) on human PBMC.

FIG. 9 shows the chemotactic effect of muKS1 and SDF-1α on THP-1 cells.

FIG. 10 shows the induction of cellular infiltrate in C3H/HeJ mice after intraperitoneal injections with muKS1 (50 μg), GV14B (50 μg) and PBS.

FIG. 11 demonstrates the induction of phosphorylation of ERK1 and ERK2 in CV1/EBNA and HeLa cell lines by huTR1a.

FIG. 12 shows the huTR1mRNA expression in HeLa cells after stimulation by muTR1, huTR1, huTGFα and PBS (100 ng/ml each).

FIG. 13 shows activation of the SRE by muTR1a in PC-12 (FIG. 13A) and HaCaT (FIG. 13B) cells.

FIG. 14 shows the inhibition of huTR1a mediated growth on HaCaT cells by an antibody to the EGF receptor.

FIG. 15A shows the nucleotide sequence of KS1 cDNA (SEQ ID NO: 464) along with the deduced amino acid sequence (SEQ ID NO: 465) using single letter code. The 5′ UTR is indicated by negative numbers. The underlined NH₂-terminal amino acids represent the predicted leader sequence and the stop codon is denoted by ***. The poly-adenylation signal is marked by a double underline. FIG. 15B shows a comparison of the complete open reading frame of KS1 (referred to in FIG. 15B as KLF-1) with its human homologue BRAK and with the mouse α-chemokines mCrg-2, mMig, mSDF-1, mBLC, mMIP2, mKC and mLIX. An additional five residues are present in KS1 and BRAK between cysteine 3 and cysteine 4 that have not previously been described for chemokines.

### DETAILED DESCRIPTION OF THE INVENTION

In one aspect, the present invention provides polynucleotides that were isolated from mammalian skin cells. The term “polynucleotide(s),” as used herein, means a single or double-stranded polymer of deoxyribonucleotide or ribonucleotide bases and includes DNA and corresponding RNA molecules, including HnRNA and mRNA molecules, both sense and anti-sense strands, and comprehends cDNA, genomic DNA and recombinant DNA, as well as wholly or partially synthesized polynucleotides. An HnRNA molecule contains introns and corresponds to a DNA molecule in a generally one-to-one manner. An mRNA molecule corresponds to an HnRNA and DNA molecule from which the introns have been excised. A polynucleotide may consist of an entire gene, or any portion thereof. Operable anti-sense polynucleotides may comprise a fragment of the corresponding polynucleotide, and the definition of “polynucleotide” therefore includes all such operable anti-sense fragments. Anti-sense polynucleotides and techniques involving anti-sense polynucleotides are well known in the art and are described, for example, in Robinson-Benion et al., Methods in Enzymol. 254: 363-375, 1995 and Kawasaki et al., Artific. Organs 20: 836-848, 1996.

Identification of genomic DNA and heterologous species DNAs can be accomplished by standard DNA/DNA hybridization techniques, under appropriately stringent conditions, using all or part of a cDNA sequence as a probe to screen an appropriate library. Alternatively, PCR techniques using oligonucleotide primers that are designed based on known genomic DNA, cDNA and protein sequences can be used to amplify and identify genomic and cDNA sequences. Synthetic DNAs corresponding to the identified sequences and variants may be produced by conventional synthesis methods. All the polynucleotides provided by the present invention are isolated and purified, as those terms are commonly used in the art.

In specific embodiments, the polynucleotides of the present invention comprise a sequence selected from the group consisting of sequences provided in SEQ ID NOS: 1-119, 198-274, 349-372, 399-405, 410-412, 416, 418-455, 464, 466-487, 510, 511 and 514-623, and variants thereof. Complements of such isolated polynucleotides, reverse complements of such isolated polynucleotides and reverse sequences of such isolated polynucleotides are also provided, together with polynucleotides comprising at least a specified number of contiguous residues (x-mers) of any of the above-mentioned polynucleotides, extended sequences corresponding to any of the above polynucleotides, antisense sequences corresponding to any of the above polynucleotides, and variants of any of the above polynucleotides, as that term is described in this specification.

The definition of the terms “complement,” “reverse complement,” and “reverse sequence,” as used herein, is best illustrated by the following example. For the sequence 5′ AGGACC 3′, the complement, reverse complement, and reverse sequence are as follows:

| | |
|---|---|
| complement | 3′ TCCTGG 5′ |
| reverse complement | 3′ GGTCCT 5′ |
| reverse sequence | 5′ CCAGGA 3′. |

Preferably, sequences that are complements of a specifically recited polynucleotide sequence are complementary over the entire length of the specific polynucleotide sequence.

Some of the polynucleotides disclosed herein are “partial” sequences, in that they do not represent a full length gene encoding a full length polypeptide. Such partial sequences may be extended by analyzing and sequencing various DNA libraries using primers and/or probes and well known hybridization and/or PCR techniques. Partial sequences may be extended until an open reading frame encoding a polypeptide, a full length polynucleotide and/or gene capable of expressing a polypeptide, or another useful portion of the genome is identified. Such extended sequences, including full length polynucleotides and genes, are described as “corresponding to” a sequence identified as one of the sequences of SEQ ID NO: 1-119, 198-274, 349-372, 399-405, 410-412, 416, 418-455, 464, 466-487, 510, 511 and 514-623, or a variant thereof, or a portion of one of the sequences of SEQ ID NO: 1-119, 198-274, 349-372, 399-405, 410-412, 416, 418-455, 464, 466-487, 510, 511 and 514-623, or a variant thereof, when the extended polynucleotide comprises an identified sequence or its variant, or an identified contiguous portion (x-mer) of one of the sequences of SEQ ID NO: 1-119, 198-274, 349-372, 399-405, 410-412, 416, 418-455, 464, 466-487, 510, 511 and 514-623, or a variant thereof. Such extended polynucleotides may have a length of from about 50 to about 4,000 nucleic acids or base pairs, and preferably have a length of less than about 4,000 nucleic acids or base pairs, more preferably yet a length of less than about 3,000 nucleic acids or base pairs, more preferably yet a length of less than about 2,000 nucleic acids or base pairs. Under some circumstances, extended polynucleotides of the present invention may have a length of less than about 1,800 nucleic acids or base pairs, preferably less than about 1,600 nucleic acids or base pairs, more preferably less than about 1,400 nucleic acids or base pairs, more preferably yet less than about 1,200 nucleic acids or base pairs, and most preferably less than about 1,000 nucleic acids or base pairs.

Similarly, RNA sequences, reverse sequences, complementary sequences, antisense sequences, and the like, corresponding to the polynucleotides of the present invention, may be routinely ascertained and obtained using the cDNA sequences identified as SEQ ID NO: 1-119, 198-274, 349-372, 399-405, 410-412, 416, 418-455, 464, 466-487, 510, 511 and 514-623.

The polynucleotides identified as SEQ ID NO: 1-119, 198-274, 349-372, 399-405, 410-412, 416, 418-455, 464, 466-487, 510, 511 and 514-623 contain open reading frames (“ORFs”), or partial open reading frames, encoding polypeptides or functional portions of polypeptides. Open reading frames may be identified using techniques that are well known in the art. These techniques include, for example, analysis for the location of known start and stop codons, most likely reading frame identification based on codon frequencies, etc. Open reading frames and portions of open reading frames may be identified in the polynucleotides of the present invention. Suitable tools and software for ORF analysis are well known in the art and include, for example, GeneWise, available from The Sanger Center, Wellcome Trust Genome Campus, Hinxton, Cambridge, CB10 1SA, United Kingdom; Diogenes, available from Computational Biology Centers, University of Minnesota, Academic Health Center, UMHG Box 43 Minneapolis Minn. 55455; and GRAIL, available from the Informatics Group, Oak Ridge National Laboratories, Oak Ridge, Tenn. TN. Once a partial open reading frame is identified, the polynucleotide may be extended in the area of the partial open reading frame using techniques that are well known in the art until the polynucleotide for the full open reading frame is identified. Thus, open reading frames encoding polypeptides and/or functional portions of polypeptides may be identified using the polynucleotides of the present invention.

Once open reading frames are identified in the polynucleotides of the present invention, the open reading frames may be isolated and/or synthesized. Expressible genetic constructs comprising the open reading frames and suitable promoters, initiators, terminators, etc., which are well known in the art, may then be constructed. Such genetic constructs may be introduced into a host cell to express the polypeptide encoded by the open reading frame. Suitable host cells may include various prokaryotic and eukaryotic cells, including plant cells, mammalian cells, bacterial cells, algae and the like.

In another aspect, the present invention provides isolated polypeptides encoded, or partially encoded, by the above polynucleotides. The term “polypeptide”, as used herein, encompasses amino acid chains of any length including full length proteins, wherein amino acid residues are linked by covalent peptide bonds. Polypeptides of the present invention may be naturally purified products, or may be produced partially or wholly using recombinant techniques. Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

The term “polypeptide encoded by a polynucleotide” as used herein, includes polypeptides encoded by a nucleotide sequence which includes a partial isolated DNA sequence of the present invention. In specific embodiments, the inventive polypeptides comprise an amino acid sequence selected from the group consisting of sequences provided in SEQ ID NOS: 120-197, 275-348, 373-398, 406-409, 413-415, 417, 456-463, 465, 488-509, 512, 513 and 624-725, as well as variants of such sequences.

Polypeptides of the present invention may be produced recombinantly by inserting a DNA sequence that encodes the polypeptide into an expression vector and expressing the polypeptide in an appropriate host. Any of a variety of expression vectors known to those of ordinary skill in the art may be employed. Expression may be achieved in any appropriate host cell that has been transformed or transfected with an expression vector containing a DNA molecule that encodes a recombinant polypeptide. Suitable host cells include prokaryotes, yeast, and higher eukaryotic cells. Preferably, the host cells employed are E. coli, insect, yeast, or a mammalian cell line such as COS or CHO. The DNA sequences expressed in this manner may encode naturally occurring polypeptides, portions of naturally occurring polypeptides, or other variants thereof.

In a related aspect, polypeptides are provided that comprise at least a functional portion of a polypeptide having an amino acid sequence selected from the group consisting of sequences provided in SEQ ID NOS: 120-197, 275-348, 373-398, 406-409, 413-415, 417, 456-463, 465, 488-509, 512-513 and 624-725, and variants thereof. As used herein, the “functional portion” of a polypeptide is that portion which contains the active site essential for affecting the function of the polypeptide, for example, the portion of the molecule that is capable of binding one or more reactants. The active site may be made up of separate portions present on one or more polypeptide chains and will generally exhibit high binding affinity. Such functional portions generally comprise at least about 5 amino acid residues, more preferably at least about 10, and most preferably at least about 20 amino acid residues.

Functional portions of a polypeptide may be identified by first preparing fragments of the polypeptide by either chemical or enzymatic digestion of the polypeptide, or by mutation analysis of the polynucleotide that encodes the polypeptide and subsequent expression of the resulting mutant polypeptides. The polypeptide fragments or mutant polypeptides are then tested to determine which portions retain biological activity, using, for example, the representative assays provided below.

Portions and other variants of the inventive polypeptides may be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems, Inc. (Foster City, Calif.), and may be operated according to the manufacturer's instructions. Variants of a native polypeptide may be prepared using standard mutagenesis techniques, such as oligonucleotide-directed site-specific mutagenesis (Kunkel, T., Proc. Natl. Acad. Sci. USA 82:488-492, 1985). Sections of DNA sequence may also be removed using standard techniques to permit preparation of truncated polypeptides.

In general, the polypeptides disclosed herein are prepared in an isolated, substantially pure, form. Preferably, the polypeptides are at least about 80% pure, more preferably at least about 90% pure, and most preferably at least about 99% pure. In certain preferred embodiments, described in detail below, the isolated polypeptides are incorporated into compositions for use in the treatment of disorders, such as skin wounds, cancers, growth and developmental defects, and inflammatory diseases.

As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 75%, more preferably at least 80%, more preferably yet at least 90%, and most preferably, at least 95% or 98% identity to a sequence of the present invention. The percentage identity may be determined using well known techniques. In one embodiment, the percentage identity is determined by aligning the two sequences to be compared as described below, determining the number of identical residues in the aligned portion, dividing that number by the total number of residues in the inventive (queried) sequence, and multiplying the result by 100.

Polynucleotides and polypeptides having a specified percentage identity to a polynucleotide or polypeptide identified in one of SEQ ID NO: 1-725 thus share a high degree of similarity in their primary structure. In addition to a specified percentage identity to a polynucleotide of the present invention, variant polynucleotides and polypeptides preferably have additional structural and/or functional features in common with a polynucleotide of the present invention. Polynucleotides having a specified degree of identity to, or capable of hybridizing to, a polynucleotide of the present invention preferably additionally have at least one of the following features: (1) they contain an open reading frame, or partial open reading frame, encoding a polypeptide, or a functional portion of a polypeptide, having substantially the same functional properties as the polypeptide, or functional portion thereof, encoded by a polynucleotide in a recited SEQ ID NO.; or (2) they contain identifiable domains in common.

Polynucleotide or polypeptide sequences may be aligned, and percentages of identical nucleotides or amino acids in a specified region may be determined against another polynucleotide or polypeptide, using computer algorithms that are publicly available. The BLASTN and FASTA algorithms, set to the default parameters described in the documentation and distributed with the algorithm, may be used for aligning and identifying the similarity of polynucleotide sequences. The alignment and similarity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia by contacting the Assistant Provost for Research, University of Virginia, PO Box 9025, Charlottesville, Va. 22906-9025. The BLASTN software is available from the National Center for Biotechnology Information (NCBI), National Library of Medicine, Building 38A, Room 8N805, Bethesda, Md. 20894. The BLASTN algorithm Version 2.0.11 [Jan. 20, 2000] set to the default parameters described in the documentation and distributed with the algorithm, is preferred for use in the determination of polynucleotide variants according to the present invention. The use of the BLAST family of algorithms, including BLASTN, BLASTP and BLASTX, is described in the publication of Altschul et al., “Gapped BLAST and PSI-BLAST: a new generation of protein database search programs,” Nucleic Acids Res. 25:3389-3402, 1997.

The following running parameters are preferred for determination of alignments and similarities using BLASTN that contribute to the E values and percentage identity for polynucleotides: Unix running command with the following default parameters: blastall-p blastn-d embldb-e 10-G 0-E 0-r 1-v 30-b 30-i queryseq-o results; and parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (BLASTN only) [Integer]; -v Number of one-line descriptions (V) [Integer]; -b Number of alignments to show (B) [Integer]; -i Query File [File In]; -o BLAST report Output File [File Out] Optional.

The following running parameters are preferred for determination of alignments and similarities using BLASTP that contribute to the E values and percentage identity of polypeptide sequences: blastall-p blastp-d swissprotdb-e 10-G 0-E 0-v 30-b 30-i queryseq-o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -v Number of one-line descriptions (v) [Integer]; -b Number of alignments to show (b) [Integer]; -I Query File [File In]; -o BLAST report Output File [File Out] Optional.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

As noted above, the percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity. By way of example, a queried polynucleotide having 220 nucleic acids has a hit to a polynucleotide sequence in the EMBL database having 520 nucleic acids over a stretch of 23 nucleotides in the alignment produced by the BLASTN algorithm using the default parameters. The 23-nucleotide hit includes 21 identical nucleotides, one gap and one different nucleotide. The percentage identity of the queried polynucleotide to the hit in the EMBL database is thus 21/220 times 100, or 9.5%. The percentage identity of polypeptide sequences may be determined in a similar fashion.

The BLASTN and BLASTX algorithms also produce “Expect” values for polynucleotide and polypeptide alignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the sequences then have a probability of 90% of being related. For sequences having an E value of 0.01 or less over aligned and matched portions, the probability of finding a match by chance in the EMBL database is 1% or less using the BLASTN algorithm. E values for polypeptide sequences may be determined in a similar fashion using various polypeptide databases, such as the SwissProt database.

According to one embodiment, “variant” polynucleotides and polypeptides, with reference to each of the polynucleotides and polypeptides of the present invention, preferably comprise sequences having the same number or fewer nucleotides or amino acids than each of the polynucleotides or polypeptides of the present invention and producing an E value of 0.01 or less when compared to the polynucleotide or polypeptide of the present invention. That is, a variant polynucleotide or polypeptide is any sequence that has at least a 99% probability of being related to the polynucleotide or polypeptide of the present invention, measured as having an E value of 0.01 or less using the BLASTN or BLASTX algorithms set at the default parameters. According to a preferred embodiment, a variant polynucleotide is a sequence having the same number or fewer nucleic acids than a polynucleotide of the present invention that has at least a 99% probability of being related to the polynucleotide of the present invention, measured as having an E value of 0.01 or less using the BLASTN algorithm set at the default parameters. Similarly, according to a preferred embodiment, a variant polypeptide is a sequence having the same number or fewer amino acids than a polypeptide of the present invention that has at least a 99% probability of being related as the polypeptide of the present invention, measured as having an E value of 0.01 or less using the BLASTP algorithm set at the default parameters.

In an alternative embodiment, variant polynucleotides are sequences that hybridize to a polynucleotide of the present invention under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents, and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The present invention also encompasses polynucleotides that differ from the disclosed sequences but that, as a consequence of the discrepancy of the genetic code, encode a polypeptide having similar enzymatic activity to a polypeptide encoded by a polynucleotide of the present invention. Thus, polynucleotides comprising sequences that differ from the polynucleotide sequences recited in SEQ ID NO: 1-119, 198-276, 349-372, 399-405, 410-412, 416, 418-455, 464, 466-487, 510, 511 and 514-623, or complements, reverse sequences, or reverse complements of those sequences, as a result of conservative substitutions are contemplated by and encompassed within the present invention. Additionally, polynucleotides comprising sequences that differ from the polynucleotide sequences recited in SEQ ID NO: 1-119, 198-276, 349-372, 399-405, 410-412, 416, 418-455, 464, 466-487, 510, 511 and 514-623, or complements, reverse complements or reverse sequences thereof, as a result of deletions and/or insertions totaling less than 10% of the total sequence length are also contemplated by and encompassed within the present invention. Similarly, polypeptides comprising sequences that differ from the polypeptide sequences recited in SEQ ID NO: 120-197, 275-348, 373-398, 406-409, 413-415, 417, 456-463, 465, 488-509, 512, 513 and 624-725 as a result of amino acid substitutions, insertions, and/or deletions totaling less than 10% of the total sequence length are contemplated by and encompassed within the present invention, provided the variant polypeptide has functional properties which are substantially the same as, or substantially similar to those of a polypeptide comprising a sequence of SEQ ID NO: 120-197, 275-348, 373-398, 406-409, 413-415, 417, 456-463, 465, 488-509, 512, 513 and 624-725.

As used herein, the term “x-mer,” with reference to a specific value of “x,” refers to a polynucleotide or polypeptide, respectively, comprising at least a specified number (“x”) of contiguous residues of: any of the polynucleotides provided in SEQ ID NO: 1-119, 198-274, 349-372, 399-405, 410-412, 416, 418-455, 464, 466-487, 510, 511 and 514-623; or any of the polypeptides set out in SEQ ID NO: 120-197, 275-348, 373-398, 406-409, 413-415, 417, 456-463, 465, 488-509, 512, 513 and 624-725. The value of x may be from about 20 to about 600, depending upon the specific sequence.

Polynucleotides of the present invention comprehend polynucleotides comprising at least a specified number of contiguous residues (x-mers) of any of the polynucleotides identified as SEQ ID NO: 1-119, 198-274, 349-372, 399-405, 410-412, 416, 418-455, 464, 466-487, 510, 511 and 514-623, or their variants. Polypeptides of the present invention comprehend polypeptides comprising at least a specified number of contiguous residues (x-mers) of any of the polypeptides identified as SEQ ID NO: 120-197, 275-348, 373-398, 406-409, 413-415, 417, 456-463, 465, 488-509, 512, 513 and 624-725. According to preferred embodiments, the value of x is at least 20, more preferably at least 40, more preferably yet at least 60, and most preferably at least 80. Thus, polynucleotides of the present invention include polynucleotides comprising a 20-mer, a 40-mer, a 60-mer, an 80-mer, a 100-mer, a 120-mer, a 150-mer, a 180-mer, a 220-mer, a 250-mer; or a 300-mer, 400-mer, 500-mer or 600-mer of a polynucleotide provided in SEQ ID NOS: 1-119, 198-274, 349-372, 399-405, 410-412, 416, 418-455, 464, 466-487, 510, 511 and 514-623, or of a variant of one of the polynucleotides provided in SEQ ID NO: 1-119, 198-274, 349-372, 399-405, 410-412, 416, 418-455, 464, 466-487, 510, 511 and 514-623. Polypeptides of the present invention include polypeptides comprising a 20-mer, a 40-mer, a 60-mer, an 80-mer, a 100-mer, a 120-mer, a 150-mer, a 180-mer, a 220-mer, a 250-mer; or a 300-mer, 400-mer, 500-mer or 600-mer of a polypeptide provided in SEQ ID NOS: 120-197, 275-348, 373-398, 406-409, 413-415, 417, 456-463, 465, 488-509, 512, 513 and 624-725, or of a variant of one of the polypeptides provided in SEQ ID NOS: 120-197, 275-348, 373-398, 406-409, 413-415, 417, 456-463, 465, 488-509, 512, 513 and 624-725.

The inventive polynucleotides may be isolated by high throughput sequencing of cDNA libraries prepared from mammalian skin cells as described below in Example 1. Alternatively, oligonucleotide probes based on the sequences provided in SEQ ID NOS: 1-119, 198-274, 349-372, 399-405, 410-412, 416, 418-455, 464, 466-487, 510, 511 and 514-623 can be synthesized and used to identify positive clones in either cDNA or genomic DNA libraries from mammalian skin cells by means of hybridization or polymerase chain reaction (PCR) techniques. Probes can be shorter than the sequences provided herein but should be at least about 10, preferably at least about 15 and most preferably at least about 20 nucleotides in length. Hybridization and PCR techniques suitable for use with such oligonucleotide probes are well known in the art (see, for example, Mullis, et al., Cold Spring Harbor Symp. Quant. Biol., 51:263, 1987; Erlich, ed., PCR Technology, Stockton Press: NY, 1989; (Sambrook, J, Fritsch, E F and Maniatis, T, eds., Molecular Cloning: A Laboratory Manual, 2nd ed., Cold Spring Harbor Laboratory Press, Cold Spring Harbor, N.Y., 1989). Positive clones may be analyzed by restriction enzyme digestion, DNA sequencing or the like.

In addition, DNA sequences of the present invention may be generated by synthetic means using techniques well known in the art. Equipment for automated synthesis of oligonucleotides is commercially available from suppliers such as Perkin Elmer/Applied Biosystems Division (Foster City, Calif.) and may be operated according to the manufacturer's instructions.

The present invention also provides fusion proteins comprising a first and a second inventive polypeptide or, alternatively, a polypeptide of the present invention and a known polypeptide, together with variants of such fusion proteins. The fusion proteins of the present invention may include a linker peptide between the first and second polypeptides.

A polynucleotide encoding a fusion protein of the present invention is constructed using known recombinant DNA techniques to assemble separate polynucleotides encoding the first and second polypeptides into an appropriate expression vector. The 3′ end of a polynucleotide encoding the first polypeptide is ligated, with or without a peptide linker, to the 5′ end of a DNA sequence polynucleotide encoding the second polypeptide so that the reading frames of the sequences are in phase to permit mRNA translation of the two polynucleotides into a single fusion protein that retains the biological activity of both the first and the second polypeptides.

A peptide linker sequence may be employed to separate the first and the second polypeptides by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may be from 1 to about 50 amino acids in length. Peptide linker sequences are not required when the first and second polypeptides have non-essential N-terminal amino acid regions that can be used to separate the functional domains and prevent steric interference.

The ligated polynucleotides encoding the fusion proteins are cloned into suitable expression systems using techniques known to those of ordinary skill in the art.

Since the polynucleotide sequences of the present invention have been derived from skin, they likely encode proteins that have important roles in growth and development of skin, and in responses of skin to tissue injury and inflammation as well as disease states. Some of the polynucleotides contain sequences that code for signal sequences, or transmembrane domains, which identify the protein products as secreted molecules or receptors. Such protein products are likely to be growth factors, cytokines, or their cognate receptors. Several of the polypeptide sequences have more than 25% similarity to known biologically important proteins and thus are likely to represent proteins having similar biological functions.

In particular, the inventive polypeptides have important roles in processes such as: induction of hair growth; differentiation of skin stem cells into specialized cell types; cell migration; cell proliferation and cell-cell interaction. The polypeptides are important in the maintenance of tissue integrity, and thus are important in processes such as wound healing. Some of the disclosed polypeptides act as modulators of immune responses, especially since immune cells are known to infiltrate skin during tissue insult causing growth and differentiation of skin cells. In addition, many polypeptides are immunologically active, making them important therapeutic targets in a whole range of disease states not only within skin, but also in other tissues of the body. Antibodies to the polypeptides of the present invention and small molecule inhibitors related to the polypeptides of the present invention may also be used for modulating immune responses and for treatment of diseases according to the present invention.

In one aspect, the present invention provides methods for using one or more of the inventive polypeptides, fusion proteins or polynucleotides to treat disorders in a patient. As used herein, a “patient” refers to any warm-blooded animal, preferably a human.

In this aspect, the polypeptide, fusion protein or polynucleotide is generally present within a pharmaceutical or immunogenic composition. Pharmaceutical compositions may comprise one or more polypeptides, each of which may contain one or more of the above sequences (or variants thereof), and a physiologically acceptable carrier. Immunogenic compositions may comprise one or more of the above polypeptides and an immunostimulant, such as an adjuvant or a liposome, into which the polypeptide is incorporated.

Alternatively, a composition of the present invention may contain DNA encoding one or more polypeptides or fusion proteins as described above, such that the polypeptide or fusion protein is generated in situ. In such compositions, the DNA may be present within any of a variety of delivery systems known to those of ordinary skill in the art, including nucleic acid expression systems, and bacterial and viral expression systems. Appropriate nucleic acid expression systems contain the necessary DNA sequences for expression in the patient (such as a suitable promoter and terminator signal). Bacterial delivery systems involve the administration of a bacterium (such as Bacillus-Calmette-Guerin) that expresses an immunogenic portion of the polypeptide on its cell surface. In a preferred embodiment, the DNA may be introduced using a viral expression system (e.g., vaccinia or other poxvirus, retrovirus, or adenovirus), which may involve the use of a non-pathogenic, or defective, replication competent virus. Techniques for incorporating DNA into such expression systems are well known in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

Routes and frequency of administration, as well as dosage, vary from individual to individual. In general, the inventive compositions may be administered by injection (e.g., intradermal, intramuscular, intravenous, or subcutaneous), intranasally (e.g., by aspiration) or orally. In general, the amount of polypeptide present in a dose (or produced in situ by the DNA in a dose) ranges from about 1 pg to about 100 mg per kg of host, typically from about 10 pg to about 1 mg per kg of host, and preferably from about 100 pg to about 1 μg per kg of host. Suitable dose sizes will vary with the size of the patient, but will typically range from about 0.1 ml to about 5 ml.

While any suitable carrier known to those of ordinary skill in the art may be employed in the compositions of this invention, the type of carrier will vary depending on the mode of administration. For parenteral administration, such as subcutaneous injection, the carrier preferably comprises water, saline, alcohol, a lipid, a wax, or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

Any of a variety of adjuvants may be employed in the immunogenic compositions of the invention to non-specifically enhance the immune response. Most adjuvants contain a substance designed to protect the antigen from rapid catabolism, such as aluminum hydroxide or mineral oil, and a non-specific stimulator of immune responses, such as lipid A, Bordetella pertussis, or Mycobacterium tuberculosis. Suitable adjuvants are commercially available as, for example, Freund's Incomplete Adjuvant and Freund's Complete Adjuvant (Difco Laboratories, Detroit, Mich.), and Merck Adjuvant 65 (Merck and Company, Inc., Rahway, N.J.). Other suitable adjuvants include alum, biodegradable microspheres, monophosphoryl lipid A, and Quil A.

The polynucleotides of the present invention may also be used as markers for tissue, as chromosome markers or tags, in the identification of genetic disorders, and for the design of oligonucleotides for examination of expression patterns using techniques well known in the art, such as the microarray technology available from Affymetrix (Santa Clara, Calif.). Partial polynucleotide sequences disclosed herein may be employed to obtain full length genes by, for example, screening of DNA expression libraries using hybridization probes or PCR primers based on the inventive sequences.

The polypeptides provided by the present invention may additionally be used in assays to determine biological activity, to raise antibodies, to isolate corresponding ligands or receptors, in assays to quantitatively determine levels of protein or cognate corresponding ligand or receptor, as anti-inflammatory agents, and in compositions for skin, connective tissue and/or nerve tissue growth or regeneration.

The isolated polynucleotides of the present invention also have utility in genome mapping, in physical mapping, and in positional cloning of genes. As detailed below, the polynucleotide sequences identified as SEQ ID NO: 1-119, 198-276, 349-372, 399-405, 410-412, 416, 418-455, 464, 466-487, 510, 511 and 514-623, and their variants, may be used to design oligonucleotide probes and primers. Oligonucleotide probes designed using the polynucleotides of the present invention may be used to detect the presence and examine the expression patterns of genes in any organism having sufficiently similar DNA and RNA sequences in their cells using techniques that are well known in the art, such as slot blot DNA hybridization techniques. Oligonucleotide primers designed using the polynucleotides of the present invention may be used for PCR amplifications. Oligonucleotide probes and primers designed using the polynucleotides of the present invention may also be used in connection with various microarray technologies, including the microarray technology of Affymetrix (Santa Clara, Calif.).

As used herein, the term “oligonucleotide” refers to a relatively short segment of a polynucleotide sequence, generally comprising between 6 and 60 nucleotides, and comprehends both probes for use in hybridization assays and primers for use in the amplification of DNA by polymerase chain reaction. An oligonucleotide probe or primer is described as “corresponding to” a polynucleotide of the present invention, including one of the sequences set out as SEQ ID NO: 1-119, 198-276, 349-372, 399-405, 410-412, 416, 418-455, 464, 466-487, 510, 511 and 514-623, or a variant thereof, if the oligonucleotide probe or primer, or its complement, is contained within one of the sequences set out as SEQ ID NO: 1-119, 198-276, 349-372, 399-405, 410-412, 416, 418-455, 464, 466-487, 510, 511 and 514-623, or a variant of one of the specified sequences. Oligonucleotide probes and primers of the present invention are substantially complementary to a polynucleotide disclosed herein.

Two single stranded sequences are said to be substantially complementary when the nucleotides of one strand, optimally aligned and compared, with the appropriate nucleotide insertions and/or deletions, pair with at least 80%, preferably at least 90% to 95% and more preferably at least 98% to 100% of the nucleotides of the other strand. Alternatively, substantial complementarity exists when a first DNA strand will selectively hybridize to a second DNA strand under stringent hybridization conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone.

In specific embodiments, the oligonucleotide probes and/or primers comprise at least about 6 contiguous residues, more preferably at least about 10 contiguous residues, and most preferably at least about 20 contiguous residues complementary to a polynucleotide sequence of the present invention. Probes and primers of the present invention may be from about 8 to 100 base pairs in length or, preferably from about 10 to 50 base pairs in length or, more preferably from about 15 to 40 base pairs in length. The probes can be easily selected using procedures well known in the art, taking into account DNA-DNA hybridization stringencies, annealing and melting temperatures, and potential for formation of loops and other factors, which are well known in the art. Tools and software suitable for designing probes and PCR primers are well known in the art and include the software program available from Premier Biosoft International, 3786 Corina Way, Palo Alto, Calif. 94303-4504. Preferred techniques for designing PCR primers are also disclosed in Dieffenbach, CW and Dyksler, GS. PCR Primer: a laboratory manual, CSHL Press: Cold Spring Harbor, N.Y., 1995.

A plurality of oligonucleotide probes or primers corresponding to a polynucleotide of the present invention may be provided in a kit form. Such kits generally comprise multiple DNA or oligonucleotide probes or primers, each probe or primer being specific for a polynucleotide sequence. Kits of the present invention may comprise one or more probes or primers corresponding to a polynucleotide of the present invention, including a polynucleotide sequence identified in SEQ ID NO: 1-119, 198-276, 349-372, 399-405, 410-412, 416, 418-455, 464, 466-487, 510, 511 and 514-623.

In one embodiment useful for high-throughput assays, the oligonucleotide probe kits of the present invention comprise multiple probes in an array format, wherein each probe is immobilized at a predefined, spatially addressable, location on the surface of a solid substrate. Array formats which may be usefully employed in the present invention are disclosed, for example, in U.S. Pat. No. 5,412,087 and 5,545,451, and PCT Publication No. WO 95/00450, the disclosures of which are hereby incorporated by reference.

The present invention further provides methods and compositions for reducing the levels and/or inhibiting the activity of an inventive polypeptide or polynucleotide. Such methods include administering a component selected from the group consisting of: antibodies, or antigen-binding fragments thereof, that specifically bind to a polypeptide of the present invention; soluble ligands that bind to an inventive polypeptide; small molecule inhibitors of the inventive polypeptides and/or polynucleotides; anti-sense oligonucleotides to the inventive polynucleotides; small interfering RNA molecules (siRNA or RNAi) that are specific for a polynucleotide or polypeptide of the present invention; and engineered soluble polypeptide molecules that bind a ligand of an inventive polypeptide but do not stimulate signaling.

Modulating the activity of a polypeptide described herein may be accomplished by reducing or inhibiting expression of the polypeptides, which can be achieved by interfering with transcription and/or translation of the corresponding polynucleotide. Polypeptide expression may be inhibited, for example, by introducing anti-sense expression vectors; by introducing anti-sense oligodeoxyribonucleotides, anti-sense phosphorothioate oligodeoxyribonucleotides, anti-sense oligoribonucleotides or anti-sense phosphorothioate oligoribonucleotides; or by other means well known in the art. All such anti-sense polynucleotides are referred to collectively herein as “anti-sense oligonucleotides”.

The anti-sense oligonucleotides disclosed herein are sufficiently complementary to the polynucleotide encoding the inventive polypeptide to bind specifically to the polynucleotide. The sequence of an anti-sense oligonucleotide need not be 100% complementary to that of the polynucleotide in order for the anti-sense oligonucleotide to be effective in the inventive methods. Rather an anti-sense oligonucleotide is sufficiently complementary when binding of the anti-sense oligonucleotide to the polynucleotide interferes with the normal function of the polynucleotide to cause a loss of utility, and when non-specific binding of the oligonucleotide to other, non-target, sequences is avoided. The present invention thus encompasses polynucleotides in an anti-sense orientation that inhibit translation of the inventive polypeptides. The design of appropriate anti-sense oligonucleotides is well known in the art. Oligonucleotides that are complementary to the 5′ end of the message, for example the 5′ untranslated sequence up to and including the AUG initiation codon, should work most efficiently at inhibiting translation. However, oligonucleotides complementary to either the 5′- or 3′-non-translated, non-coding, regions of the targeted polynucleotide can be used.

Cell permeation and activity of anti-sense oligonucleotides can be enhanced by appropriate chemical modifications, such as the use of phenoxazine-substituted C-5 propynyl uracil oligonucleotides (Flanagan et al., Nat. Biotechnol. 17:48-52 (1999)) or 2′-O-(2-methoxy) ethyl (2′-MOE)-oligonucleotides (Zhang et al., Nat. Biotechnol. 18:862-867 (2000)). The use of techniques involving anti-sense oligonucleotides is well known in the art and is described, for example, in Robinson-Benion et al., Methods in Enzymol. 254:363-375 (1995) and Kawasaki et al., Artific. Organs 20:836-848 (1996).

Expression of a polypeptide of the present invention may also be specifically suppressed by methods such as RNA interference (RNAi). A review of this technique is found in Science, 288:1370-1372, 2000. Briefly, traditional methods of gene suppression, employing anti-sense RNA or DNA, operate by binding to the reverse sequence of a gene of interest such that binding interferes with subsequent cellular processes and therefore blocks synthesis of the corresponding protein. RNAi also operates on a post-translational level and is sequence specific, but suppresses gene expression far more efficiently. Exemplary methods for controlling or modifying gene expression are provided in WO 99/49029, WO 99/53050 and WO01/75164, the disclosures of which are hereby incorporated by reference. In these methods, post-transcriptional gene silencing is brought about by a sequence-specific RNA degradation process which results in the rapid degradation of transcripts of sequence-related genes. Studies have shown that double-stranded RNA may act as a mediator of sequence-specific gene silencing (see, for example, Montgomery and Fire, Trends in Genetics, 14:255-258, 1998). Gene constructs that produce transcripts with self-complementary regions are particularly efficient at gene silencing.

It has been demonstrated that one or more ribonucleases specifically bind to and cleave double-stranded RNA into short fragments. The ribonuclease(s) remains associated with these fragments, which in turn specifically bind to complementary mRNA, i.e. specifically bind to the transcribed mRNA strand for the gene of interest. The mRNA for the gene is also degraded by the ribonuclease(s) into short fragments, thereby obviating translation and expression of the gene. Additionally, an RNA-polymerase may act to facilitate the synthesis of numerous copies of the short fragments, which exponentially increases the efficiency of the system. A unique feature of RNAi is that silencing is not limited to the cells where it is initiated. The gene-silencing effects may be disseminated to other parts of an organism.

The polynucleotides of the present invention may thus be employed to generate gene silencing constructs and/or gene-specific self-complementary, double-stranded RNA sequences that can be delivered by conventional art-known methods. A gene construct may be employed to express the self-complementary RNA sequences. Alternatively, cells are contacted with gene-specific double-stranded RNA molecules, such that the RNA molecules are internalized into the cell cytoplasm to exert a gene silencing effect. The double-stranded RNA must have sufficient homology to the targeted gene to mediate RNAi without affecting expression of non-target genes. The double-stranded DNA is at least 20 nucleotides in length, and is preferably 21-23 nucleotides in length. Preferably, the double-stranded RNA corresponds specifically to a polynucleotide of the present invention. The use of small interfering RNA (siRNA) molecules of 21-23 nucleotides in length to suppress gene expression in mammalian cells is described in WO 01/75164. Tools for designing optimal inhibitory siRNAs include that available from DNAengine Inc. (Seattle, Wash.).

One RNAi technique employs genetic constructs within which sense and anti-sense sequences are placed in regions flanking an intron sequence in proper splicing orientation with donor and acceptor splicing sites. Alternatively, spacer sequences of various lengths may be employed to separate self-complementary regions of sequence in the construct. During processing of the gene construct transcript, intron sequences are spliced-out, allowing sense and anti-sense sequences, as well as splice junction sequences, to bind forming double-stranded RNA. Select ribonucleases then bind to and cleave the double-stranded RNA, thereby initiating the cascade of events leading to degradation of specific mRNA gene sequences, and silencing specific genes.

As used herein, the phrase “contacting a population of cells with a genetic construct, anti-sense oligonucleotide or RNA molecule” includes any means of introducing a nucleic acid molecule into any portion of one or more cells by any method compatible with cell viability and known to those of ordinary skill in the art. The cell or cells may be contacted in vivo, ex vivo, in vitro, or any combination thereof.

For in vivo uses, a genetic construct, anti-sense oligonucleotide or RNA molecule may be administered by various art-recognized procedures. See, e.g., Rolland, Crit. Rev. Therap. Drug Carrier Systems 15:143-198 (1998), and cited references. Both viral and non-viral delivery methods have been used for gene therapy. Useful viral vectors include, for example, adenovirus, adeno-associated virus (AAV), retrovirus, vaccinia virus and avian poxvirus. Improvements have been made in the efficiency of targeting genes to tumor cells with adenoviral vectors, for example, by coupling adenovirus to DNA-polylysine complexes and by strategies that exploit receptor-mediated endocytosis for selective targeting. See, e.g., Curiel et al., Hum. Gene Ther., 3:147-154 (1992); and Cristiano and Curiel, Cancer Gene Ther. 3:49-57 (1996). Non-viral methods for delivering polynucleotides are reviewed in Chang & Seymour, (Eds) Curr. Opin. Mol. Ther., vol. 2 (2000). These methods include contacting cells with naked DNA, cationic liposomes, or polyplexes of polynucleotides with cationic polymers and dendrimers for systemic administration (Chang & Seymour, Ibid.). Liposomes can be modified by incorporation of ligands that recognize cell-surface receptors and allow targeting to specific receptors for uptake by receptor-mediated endocytosis. See, for example, Xu et al., Mol. Genet. Metab., 64:193-197 (1998); and Xu et al., Hum. Gene Ther., 10:2941-2952 (1999).

Tumor-targeting bacteria, such as Salmonella, are potentially useful for delivering genes to tumors following systemic administration (Low et al., Nat. Biotechnol. 17:37-41 (1999)). Bacteria can be engineered ex vivo to penetrate and to deliver DNA with high efficiency into mammalian epithelial cells in vivo and in vitro. See, e.g., Grillot-Courvalin et al., Nat. Biotechnol. 16:862-866 (1998). Degradation-stabilized oligonucleotides may be encapsulated into liposomes and delivered to patients by injection either intravenously or directly into a target site. Alternatively, retroviral or adenoviral vectors, or naked DNA expressing anti-sense RNA for the inventive polypeptides, may be delivered into patient's cells in vitro or directly into patients in vivo by appropriate routes. Suitable techniques for use in such methods are well known in the art.

The present invention further provides binding agents, such as antibodies and antigen-binding fragments thereof, that specifically bind to a polypeptide disclosed herein, or to a portion or variant thereof. A binding agent is said to “specifically bind” to an inventive polypeptide if it reacts at a detectable level with the polypeptide, and does not react detectably with unrelated polypeptides under similar conditions Any agent that satisfies this requirement may be a binding agent. For example, a binding agent may be a ribosome, with or without a peptide component, an RNA molecule, or a polypeptide. In a preferred embodiment, a binding agent is an antibody or an antigen-binding fragment thereof. The ability of an antibody, or antigen-binding fragment thereof, to specifically bind to a polypeptide can be determined, for example, in an ELISA assay using techniques well known in the art.

An “antigen-binding site,” or “antigen-binding fragment” of an antibody refers to the part of the antibody that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the inventive polypeptide is initially injected into any of a wide variety of mammals (e.g., mice, rats, rabbits, sheep or goats). The polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the inventive polypeptide may then be purified from such antisera by, for example, affinity chromatography using the polypeptide coupled to a suitable solid support.

Monoclonal antibodies specific for an inventive polypeptide may be prepared using the technique of Kohler and Milstein, Eur. J. Immunol. 6:511-519, 1976, and improvements thereto. These methods involve the preparation of immortal cell lines capable of producing antibodies having the desired specificity. Such cell lines may be produced from spleen cells obtained from an animal immunized as described above. The spleen cells are then immortalized by, for example, fusion with a myeloma cell fusion partner, preferably one that is syngeneic with the immunized animal. A variety of fusion techniques well known in the art may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may then be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of molecules are known in the art that comprise antigen-binding sites capable of exhibiting the binding properties of an antibody molecule. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment, which comprises both antigen-binding sites. An “Fv” fragment can be produced by preferential proteolytic cleavage of an IgM, IgG or IgA immunoglobulin molecule, but are more commonly derived using recombinant techniques known in the art. The Fv fragment includes a non-covalent V_{H}::V_{L} heterodimer including an antigen-binding site which retains much of the antigen recognition and binding capabilities of the native antibody molecule (Inbar et al. Proc. Nat. Acad. Sci. USA 69:2659-2662 (1972); Hochman et al. Biochem 15:2706-2710 (1976); and Ehrlich et al. Biochem 19:4091-4096 (1980)).

The present invention further encompasses humanized antibodies that specifically bind to an inventive polypeptide. A number of humanized antibody molecules comprising an antigen-binding site derived from a non-human immunoglobulin have been described, including chimeric antibodies having rodent V regions and their associated CDRs fused to human constant domains (Winter et al. Nature 349:293-299 (1991); Lobuglio et al. Proc. Nat. Acad. Sci. USA 86:4220-4224 (1989); Shaw et al. J. Immunol. 138:4534-4538 (1987); and Brown et al. Cancer Res. 47:3577-3583 (1987)); rodent CDRs grafted into a human supporting FR prior to fusion with an appropriate human antibody constant domain (Riechmann et al. Nature 332:323-327 (1988); Verhoeyen et al. Science 239:1534-1536 (1988); and Jones et al. Nature 321:522-525 (1986)); and rodent CDRs supported by recombinantly veneered rodent FRs (European Patent Publication No. 519,596, published Dec. 23, 1992). These “humanized” molecules are designed to minimize unwanted immunological responses towards rodent antihuman antibody molecules which limit the duration and effectiveness of therapeutic applications of those moieties in human recipients.

The following Examples are offered by way of illustration and not by way of limitation.

### EXAMPLE 1

### Isolation of cDNA Sequences form Skin Cell Expression Libraries

The cDNA sequences of the present invention were obtained by high-throughput sequencing of cDNA expression libraries constructed from specialized rodent or human skin cells as shown in Table 1.

TABLE 1

| Library | Skin cell type | Source |
|---|---|---|
| DEPA | dermal papilla | rat |
| SKTC | keratinocytes | human |
| HNFF | neonatal foreskin fibroblast | human |
| MEMS | embryonic skin | mouse |
| KSCL | keratinocyte stem cell | mouse |
| TRAM | transit amplifying cells | mouse |
| MFSE | epidermis | mouse |
| HLEA | small epithelial airway cells | human |
| HLEB | small epithelial airway cells | human |
| HNKA | NK cells | human |

These cDNA libraries were prepared as described below.

cDNA Library from Dermal Papilla (DEPA)

Dermal papilla cells from rat hair vibrissae (whiskers) were grown in culture and the total RNA extracted from these cells using established protocols. Total RNA, isolated using TRIzol Reagent (BRL Life Technologies, Gaithersburg, Md.), was used to obtain mRNA using a Poly(A) Quik mRNA isolation kit (Stratagene, La Jolla, Calif.), according to the manufacturer's specifications. A cDNA expression library was then prepared from the mRNA by reverse transcriptase synthesis using a Lambda ZAP cDNA library synthesis kit (Stratagene).

cDNA Library from Keratinocytes (SKTC)

Keratinocytes obtained from human neonatal foreskins (Mitra, R and Nikoloff, B in Handbook of Keratinocyte Methods, pp. 17-24, 1994) were grown in serum-free KSFM (BRL Life Technologies) and harvested along with differentiated cells (10⁸ cells). Keratinocytes were allowed to differentiate by addition of fetal calf serum at a final concentration of 10% to the culture medium and cells were harvested after 48 hours. Total RNA was isolated from the two cell populations using TRIzol Reagent (BRL Life Technologies) and used to obtain mRNA using a Poly(A) Quik mRNA isolation kit (Stratagene). cDNAs expressed in differentiated keratinocytes were enriched by using a PCR-Select cDNA Subtraction Kit (Clontech, Palo Alto, Calif.). Briefly, mRNA was obtained from either undifferentiated keratinocytes (“driver mRNA”) or differentiated keratinocytes (“tester mRNA”) and used to synthesize cDNA. The two populations of cDNA were separately digested with RsaI to obtain shorter, blunt-ended molecules. Two tester populations were created by ligating different adaptors at the cDNA ends and two successive rounds of hybridization were performed with an excess of driver cDNA. The adaptors allowed for PCR amplification of only the differentially expressed sequences which were then ligated into T-tailed pBluescript (Hadjeb, N and Berkowitz, G A, BioTechniques 20:20-22 1996), allowing for a blue/white selection of cells containing vector with inserts. White cells were isolated and used to obtain plasmid DNA for sequencing.

cDNA Library from Human Neonatal Fibroblasts (HNFF)

Human neonatal fibroblast cells were grown in culture from explants of human neonatal foreskin and the total RNA extracted from these cells using established protocols. Total RNA, isolated using TRIzol Reagent (BRL Life Technologies, Gaithersburg, Md.), was used to obtain mRNA using a Poly(A) Quik mRNA isolation kit (Stratagene, La Jolla, Calif.), according to the manufacturer's specifications. A cDNA expression library was then prepared from the mRNA by reverse transcriptase synthesis using a Lambda ZAP cDNA library synthesis kit (Stratagene).

cDNA Library from Mouse Embryonic Skin (MEMS)

Embryonic skin was micro-dissected from day 13 post coitum Balb/c mice. Embryonic skin was washed in phosphate buffered saline and mRNA directly isolated from the tissue using the Quick Prep Micro mRNA purification kit (Pharmacia, Sweden). The mRNA was then used to prepare cDNA libraries as described above for the DEPA library.

cDNA Library from Mouse Stem Cells (KSCL) and Transit Amplifying (TRAM) Cells

Pelts obtained from 1-2 day postpartum neonatal Balb/c mice were washed and incubated in trypsin (BRL Life Technologies) to separate the epidermis from the dermis. Epidermal tissue was disrupted to disperse cells, which were then resuspended in growth medium and centrifuged over Percoll density gradients prepared according to the manufacturer's protocol (Pharmacia, Sweden). Pelleted cells were labeled using Rhodamine 123 (Bertoncello I, Hodgson G S and Bradley T R, Exp Hematol. 13:999-1006, 1985), and analyzed by flow cytometry (Epics Elite Coulter Cytometry, Hialeah, Fla.). Single cell suspensions of rhodamine-labeled murine keratinocytes were then labeled with a cross reactive anti-rat CD29 biotin monoclonal antibody (Pharmingen, San Diego, Calif.; clone Ha2/5). Cells were washed and incubated with anti-mouse CD45 phycoerythrin conjugated monoclonal antibody (Pharmingen; clone 30F11.1, 10 ug/ml) followed by labeling with streptavidin spectral red (Southern Biotechnology, Birmingham, Ala.). Sort gates were defined using listmode data to identify four populations: CD29 bright rhodamine dull CD45 negative cells; CD29 bright rhodamine bright CD45 negative cells; CD29 dull rhodamine bright CD45 negative cells; and CD29 dull rhodamine dull CD45 negative cells. Cells were sorted, pelleted and snap frozen prior to storage at −80° C. This protocol was followed multiple times to obtain sufficient cell numbers of each population to prepare cDNA libraries. Skin stem cells and transit amplifying cells are known to express CD29, the integrin β1 chain. CD45, a leukocyte specific antigen, was used as a marker for cells to be excluded in the isolation of skin stem cells and transit amplifying cells. Keratinocyte stem cells expel the rhodamine dye more efficiently than transit amplifying cells. The CD29 bright, rhodamine dull, CD45 negative population (putative keratinocyte stem cells; referred to as KSCL), and the CD29 bright, rhodamine bright, CD45 negative population (keratinocyte transit amplifying cells; referred to as TRAM) were sorted and mRNA was directly isolated from each cell population using the Quick Prep Micro mRNA purification kit (Pharmacia, Sweden). The mRNA was then used to prepare cDNA libraries as described above for the DEPA library.

cDNA Library from Epithelial Cells (MFSE)

Skin epidermis was removed from flaky skin fsn −/− mice (The Jackson Laboratory, Bar Harbour, Me.), the cells dissociated and the resulting single cell suspension placed in culture. After four passages, the cells were harvested. Total RNA, isolated using TRIzol Reagent (BRL Life Technologies, Gaithersburg, Md.), was used to obtain mRNA using a Poly(A)Quik mRNA isolation kit (Stratagene, La Jolla, Calif.), according to the manufacturer's specifications. A cDNA expression library (referred to as the MFSE library) was then prepared from the mRNA by Reverse Transcriptase synthesis using a Lambda ZAP Express cDNA library synthesis kit (Stratagene, La Jolla, Calif.).

cDNA Libraries from Human Small Airway Epithelial Cells (HLEA and HLEB)

Human small airway epithelium cells SAEC (Cell line number CC-2547, Clonetics Normal Human Cell Systems, Cambrex Corporation, East Rutherford N.J.) transformed with human papilloma virus E6E7 that was infected with the bacterium Yersinia enterocolitica (ATCC No. 51871, American Type Culture Collection, Manassas Va.) and the long form of the Respiratory Syncytial Virus (RSV, ATCC No. VR26), were used as source of RNA to construct the libraries called HLEA and HLEB. Cells from the twelfth passage of SAEC cells were infected with Y. enterocolitica for 2 hours at an initial seed of 12.5 bacteria per cell. The cells were disinfected with gentamycin (100 μg/ml) for 2 hours and harvested 4 hours after infection. The cells were then infected with RSV at a moiety of infection of 0.7 for 1 hour and incubated for 6 and 24 hours. Cells were harvested and the RNA extracted following standard protocols.

Total RNA, isolated using TRIzol Reagent (BRL Life Technologies, Gaithersburg, Md.), was used to obtain mRNA using a Poly(A) Quik mRNA isolation kit (Stratagene, La Jolla, Calif.), according to the manufacturer's specifications. Two cDNA expression libraries were then prepared from the mRNA by reverse transcriptase synthesis using a Lambda ZAP cDNA library synthesis kit (Stratagene).

cDNA Library from Epithelial Cells (HNKA)

The subtracted cDNA library (HNKA) from human natural killer (NK) cells was constructed as follows. A NK library was first constructed using pooled RNA extracted from primary NK cells from multiple donors, stimulated for 4 or 20 hours with IL-2 (10 ng/ml), IL-12 (1 ng/ml), IL-15 (50 ng/ml), interferon alpha (IFN-α; 1,000 U/ml) immobilized anti-CD16 or immobilized anti-NAIL antibody, or from unstimulated cells. RNA was extracted following standard procedures. cDNA was prepared using a TimeSaver kit (Pharmacia, Uppsala, Sweden) following the manufacturer's protocol. The cDNA was ligated to BglII adaptors and size-selected using cDNA sizing columns (Gibco BRL, Gaithersburg Md.). The size-selected NK cDNA was ligated into a pDc 409 vector and transformed into E. coli DH105 cells. Single-stranded DNA was prepared from the plasmid library using a helper phage (Stratagene)

A second cDNA library (referred to as FF cDNA library) was constructed using fetal foreskin tissue. RNA was extracted and cDNA prepared following standard protocols. The cDNA was ligated into the plasmid pBluescript following standard protocols. 10 μg of the FF cDNA library was linearized with the restriction endonuclease NotI and used as template to synthesize biotin-labeled cRNA using SP6 polymerase.

The subtracted NK cell library (HNKA) was constructed as follows. The biotinylated FF cRNA was mixed with the NK library, ethanol precipitated and resuspended in 5 μl buffer (50 mM HEPES pH 7.4, 10 mM EDTA, 1.5 M NaCl, 0.2% SDS). After addition of 5 μl formamide and heating to 95° for 1 min, the material was left to hybridize for 24 hours at 42° C. 90 μl of 10 mM HEPES pH 7.3, 1 mM EDTA and 15 μl streptavidin was added followed by an incubation for 20 min at 50° C. This step was repeated again after extraction with phenol/chloroform.

To the final extracted aqueous phase, the following were added: NaCl to 0.2 M, 1 μl glycogen and 2 volumes of ethanol. After an overnight precipitation at −20° C., the DNA was pelleted and resuspended in 10 μl water. A second round of subtraction was performed as above and the DNA transformed into E. coli DH105.

cDNA sequences were obtained by high-throughput sequencing of the cDNA libraries described above using a Perkin Elmer/Applied Biosystems Division Prism 377 sequencer.

### EXAMPLE 2

### Characterization of Isolated cDNA Sequences

The isolated cDNA sequences were compared to sequences in the EMBL DNA database using the computer algorithms FASTA and/or BLASTN. The corresponding protein sequences (DNA translated to protein in each of 6 reading frames) were compared to sequences in the SwissProt database using the computer algorithms FASTX and/or BLASTX. Comparisons of DNA sequences provided in SEQ ID NO: 1-119 to sequences in the EMBL DNA database (using FASTA) and amino acid sequences provided in SEQ ID NO: 120-197 to sequences in the SwissProt database (using FASTX) were made as of Mar. 21, 1998. Comparisons of DNA sequences provided in SEQ ID NO: 198-274 to sequences in the EMBL DNA database (using BLASTN) and amino acid sequences provided in SEQ ID NO: 275-348 to sequences in the SwissProt database (using BLASTP) were made as of Oct. 7, 1998. Comparisons of DNA sequences provided in SEQ ID NO: 349-372 to sequences in the EMBL DNA database (using BLASTN) and amino acid sequences provided in SEQ ID NO: 373-398 to sequences in the SwissProt database (using BLASTP) were made as of Jan. 23, 1999. Comparisons of polynucleotide sequences provided in SEQ ID NO: 418-455 and 466-487 to sequences in the EMBL DNA database (using BLASTN) and polypeptide sequences provided in SEQ ID NO: 456-463 and 488-509 to sequences in the SwissProt database (using BLASTP) were made as of Apr. 23, 2000. Comparisons of polynucleotide sequences provided in SEQ ID NO: 510 and 511 to sequences in the EMBL DNA database (using BLASTN) and polypeptide sequences provided in SEQ ID NO: 512 and 513 to sequences in the SwissProt database (using BLASTP) were made as of Jul. 11, 2000. Comparisons of polynucleotide sequences provided in SEQ ID NO: 514-623 to sequences in the EMBL66−HTGs+ENSEMBL (May 1, 2001) DNA database (using BLASTN) and polypeptide sequences provided in SEQ ID NO: 624-725 to sequences in the SP_TR_NRDB+ENSEMBL (Apr. 30, 2001) database (using BLASTP) were made as of May 16, 2001.

Isolated cDNA sequences and their corresponding polypeptide sequences were computer analyzed for the presence of signal sequences identifying secreted molecules. Isolated cDNA sequences that have a signal sequence at a putative start site within the sequence are provided in SEQ ID NO: 1-44, 198-238, 349-358, 399, 418-434, 440-449 and 466-471, 516, 519, 520, 523-527, 531, 532, 535-537, 548, 555, 574-580, 585-587, 589, 593, 595, 596, 598-601, 605-607, 609, 612, 613, 615, 616 and 622. The cDNA sequences of SEQ ID NO: 1-6, 198-199, 349-352, 354, 356-358,419-428, 430-433, 440-444, 446-448, 466, 468-470, 519, 520, 523, 524, 529, 531, 532, 535-537, 579, 585, 587, 598, 605, 609, 613 and 622 were determined to have less than 75% identity (determined as described above), to sequences in the EMBL database using the computer algorithms FASTA or BLASTN, as described above. The polypeptide sequences of SEQ ID NO: 120-125, 275-276, 373-380, 382, 456, 457, 460-462, 488-493, 633, 637, 642, 683, 685, 691, 693, 703, 706, 710, 714, 717, 718, 720, 721 and 725 were determined to have less than 75% identity (determined as described above) to sequences in the SwissProt database using the computer algorithms FASTX or BLASTP, as described above.

Further sequencing of some of the isolated partial cDNA sequences resulted in the isolation of the full-length cDNA sequences provided in SEQ ID NOS: 7-14, 200-231, 372, 418-422, 441-448, 514, 516, 557-561, 567, 568, 619 and 621. The polypeptide sequences encoded by the cDNA sequences of SEQ ID NO: 7-14, 200-231, 372, 514, 516, 557-561, 567, 568, 619 and 621 are provided in SEQ ID NOS: 126-133, 277-308, 396, 624, 626, 666-669, 674 and 724, respectively. The cDNA sequences of SEQ ID NO: 418-422 encode the same amino acid sequences as the cDNA sequences of SEQ ID NO: 7 and 11-14, namely SEQ ID NO: 126 and 130-133, respectively. Comparison of the full-length cDNA sequences with those in the EMBL database using the computer algorithm FASTA or BLASTN, as described above, revealed less than 75% identity (determined as described above) to known sequences, except for the polynucleotides in SEQ ID NOS: 516, 560 and 619. Comparison of the amino acid sequences provided in SEQ ID NOS: 126-133, 277-308, 666, 668, 669 and 724 with those in the SwissProt database using the computer algorithms FASTX or BLASTP, as described above, revealed less than 75% identity (determined as described above) to known sequences.

Comparison of the polypeptide sequences corresponding to the cDNA sequences of SEQ ID NOS: 15-23 with those in the EMBL database using the computer algorithm FASTA database showed less than 75% identity (determined as described above) to known sequences. These polypeptide sequences are provided in SEQ ID NOS: 134-142.

Further sequencing of some of the isolated partial cDNA sequences resulted in the isolation of full-length cDNA sequences provided in SEQ ID NOS: 24-44, 232-238, 423-434, 449, 466, 468-470, 475, 476 and 484. The polypeptide sequences encoded by the cDNA sequences of SEQ ID NO: 24-44, 232-238, 429, 466, 468-470, 475, 476 and 484 are provided in SEQ ID NOS: 143-163, 309-315, 456, 488, 490-492, 497, 498 and 506, respectively. The cDNA sequences of SEQ ID NO: 423-428, 430-434 and 449 encode the same polypeptide sequences as the cDNA sequences of SEQ ID NO: 27-29, 34, 35, 37, 40-44 and 238, namely SEQ ID NO: 146-148, 153, 154, 156, 159-163 and 315, respectively. These polypeptide sequences were determined to have less than 75% identity, determined as described above to known sequences in the SwissProt database using the computer algorithm FASTX.

Isolated cDNA sequences having less than 75% identity to known expressed sequence tags (ESTs) or to other DNA sequences in the public database, or whose corresponding polypeptide sequence showed less than 75% identity to known protein sequences, were computer analyzed for the presence of transmembrane domains coding for putative membrane-bound molecules. Isolated cDNA sequences that have one or more transmembrane domain(s) within the sequence are provided in SEQ ID NOS: 45-63, 239-253, 359-364, 400-402, 435, 436, 450-452, 455, 470-472, 542, 553-555, 573, 576, 581, 592, 593, 595 and 606. The cDNA sequences of SEQ ID NOS: 45-48, 239-249, 359-361, 363, 450, 451, 455, 472, 473, 553-555, 573, 576 and 592 were found to have less than 75% identity (determined as described above) to sequences in the EMBL database, using the FASTA or BLASTN computer algorithms. The polypeptide sequences encoded by the cDNA sequences of SEQ ID NO: 45-48, 239-249, 359-361, 363, 450, 451, 472, 473, 553-555, 573 and 606 (provided in SEQ ID NOS: 164-167, 316-326, 383, 385-388, 407-408, 460, 461, 494, 495, 662, 663, 664, 679, 682 and 711 respectively) were found to have less than 75% identity, determined as described above, to sequences in the SwissProt database using the FASTX or BLASTP database. The cDNA sequence of SEQ ID NO: 455 encodes the same polypeptide sequence as the cDNA sequence of SEQ ID NO: 359, namely SEQ ID NO: 383.

Comparison of the polypeptide sequences corresponding to the cDNA sequences of SEQ ID NOS: 49-63, 250-253, 436 and 452 with those in the SwissProt database showed less than 75% identity (determined as described above) to known sequences. These polypeptide sequences are provided in SEQ ID NOS: 168-182, 327-330, 457 and 462, respectively.

Using automated search programs to screen against sequences coding for molecules reported to be of therapeutic and/or diagnostic use, some of the cDNA sequences isolated as described above in Example 1 were determined to encode polypeptides that are family members of known protein families. A family member is herein defined to have at least 25% identity in the translated polypeptide to a known protein or member of a protein family. These cDNA sequences are provided in SEQ ID NOS: 64-76, 254-264, 365-369, 403, 437-439, 453, 454, 475-487, 510, 511, 514-527, 529-531, 533-536, 538-546, 548, 549, 553-559, 562, 564, 565, 567, 569-575, 577-589, 591-602, 604-612, 616-618, 621 and 622. The polypeptide sequences encoded by the cDNA sequences of SEQ ID NO: 64-76, 254-264, 365-369, 403, 438, 439, 453, 475-487, 510 and 511, 514-527, 529-531, 533-536, 538-546, 548, 549, 553-559, 562, 564, 565, 567, 569-575, 577-589, 591-602, 604-612, 616-618, 621 and 622 are provided in SEQ ID NOS: 183-195, 331-341, 389-393, 409, 458, 459, 463, 497-509, 624-637, 639-641, 643-646, 648-656, 658, 659, 662-668, 670, 672-681, 683-707, 709-717 and 721-725, respectively. The cDNA sequences of SEQ ID NO: 437 and 454 encode the same amino acid sequences as the cDNA sequences of SEQ ID NO: 68 and 262, namely SEQ ID NO: 187 and 339, respectively. The cDNA sequences of SEQ ID NOS: 64-68, 254-264, 365-369, 437-439, 453, 454, 475-478, 480-482, 484, 485, 487, 511, 514, 515, 517-520, 522, 523, 525, 529-531, 535, 536, 538, 541, 544-546, 549, 553-559, 564, 565, 567, 569-573, 579, 587, 588, 592, 597, 598, 602, 604, 605, 608-611, 617, 621 and 622 show less than 75% identity (determined as described above) to sequences in the EMBL database using the FASTA or BLASTN computer algorithms. Similarly, the amino acid sequences of SEQ ID NOS: 183-195, 331-341, 389-393, 458, 459, 463, 497, 498, 503-505, 507-509, 512, 513, 628, 632, 633, 637, 640, 655, 662-666, 668, 672, 673, 676, 679, 683, 685, 688, 691, 693, 694, 702, 703, 706, 707, 710, 711, 713, 714, 717, 721, 722 and 725 show less than 75% identity to sequences in the SwissProt database.

The isolated cDNA sequences encode proteins that influence the growth, differentiation and activation of several cell types, and that may usefully be developed as agents for the treatment and diagnosis of skin wounds, cancers, growth and developmental defects, and inflammatory disease. The utility for certain of the proteins of the present invention, based on similarity to known proteins, is provided in Table 2 below, together with the location of signal peptides and transmembrane domains for certain of the inventive sequences.

TABLE 2
FUNCTIONS OF NOVEL PROTEINS

| P/N | A/A | |
|---|---|---|
| SEQ | SEQ | |
| ID | ID | |
| NO: | NO. | SIMILARITY TO KNOWN PROTEINS; FUNCTION |
| 64, | 183, | Slit, a secreted molecule required for central nervous |
| 372 | 396 | system development |
| 65 | 184 | Immunoglobulin receptor family. About 40% of leukocyte |
| | | membrane polypeptides contain immunoglobulin |
| | | superfamily domains |
| 66, | 185, | RIP protein kinase, a serine/threonine kinase that contains a |
| 403, | 409, | death domain to mediate apoptosis |
| 510 | 512 | |
| 67 | 186 | Extracellular protein with epidermal growth factor domain |
| | | capable of stimulating fibroblast proliferation |
| 68, | 187 | Transforming growth factor alpha, a protein which binds |
| 437 | | epidermal growth factor receptor and stimulates growth and |
| | | mobility of keratinocytes |
| 69 | 188 | DRS protein which has a secretion signal component and |
| | | whose expression is suppressed in cells transformed by |
| | | oncogenes |
| 70 | 189 | A33 receptor with immunoglobulin-like domains and is |
| | | expressed in greater than 95% of colon tumors |
| 71 | 190 | Interleukin-12 alpha subunit, component of a cytokine that |
| | | is important in the immune defense against intracellular |
| | | pathogens. IL-12 also stimulates proliferation and |
| | | differentiation of TH1 subset of lymphocytes |
| 72 | 191 | Tumor Necrosis Factor receptor family of proteins that are |
| | | involved in the proliferation, differentiation and death of |
| | | many cell types including B and T lymphocytes. |
| 73 | 192 | Epidermal growth factor family proteins which stimulate |
| | | growth and mobility of keratinocytes and epithelial cells. |
| | | EGF is involved in wound healing. It also inhibits gastric |
| | | acid secretion. |
| 74 | 193 | Fibronectin Type III receptor family. The fibronectin III |
| | | domains are found on the extracellular regions of cytokine |
| | | receptors |
| 75 | 194 | Serine/threonine kinases (STK2_HUMAN) which |
| | | participate in cell cycle progression and signal transduction |
| 76 | 195 | Immunoglobulin receptor family |
| 254 | 331 | Receptor with immunoglobulin-like domains and homology |
| | | to A33 receptor which is expressed in greater than 95% of |
| | | colon tumors |
| 255 | 332 | Epidermal growth factor family proteins which stimulate |
| | | growth and mobility of keratinocytes and epithelial cells. |
| | | EGF is involved in wound healing. It also inhibits gastric |
| | | acid secretion. |
| 256 | 333 | Serine/threonine kinases (STK2_HUMAN) which |
| | | participate in cell cycle progression and signal transduction |
| 257 | 334 | Contains protein kinase and ankyrin domains. Possible role |
| | | in cellular growth and differentiation. |
| 258 | 335 | Notch family proteins which are receptors involved in |
| | | cellular differentiation. |
| 259 | 336 | Extracellular protein with epidermal growth factor domain |
| 260, | 337, | Fibronectin Type III receptor family. The fibronectin III |
| 453 | 463 | domains are found on the extracellular regions of cytokine |
| | | receptors. |
| 261 | 338 | Immunoglobulin receptor family |
| 262 | 339 | ADP/ATP transporter family member containing a calcium |
| | | binding site. |
| 263 | 340 | Mouse CXC chemokine family members are regulators of |
| | | epithelial, lymphoid, myeloid, stromal and neuronal cell |
| | | migration and cancers, agents for the healing of cancers, |
| | | neuro-degenerative diseases, wound healing, inflammatory |
| | | autoimmune diseases like psoriasis, asthma, Crohus disease |
| | | and as agents for the prevention of HTV-1 of leukocytes |
| 264 | 341 | Nucleotide-sugar transporter family member. |
| 365 | 389 | Transforming growth factor betas (TGF-betas) are secreted |
| | | covalently linked to latent TGF-beta-binding proteins |
| | | (LTBPs). LTBPs are deposited in the extracellular matrix |
| | | and play a role in cell growth or differentiation. |
| 366 | 390 | htegrins are Type I membrane proteins that function as |
| | | laminin and collagen receptors and play a role in cell |
| | | adhesion. |
| 367 | 391 | Integrins are Type I membrane proteins that function as |
| | | laminin and collagen receptors and play a role in cell |
| | | adhesion. |
| 368 | 392 | Cell wall protein precursor. Are involved in cellular growth |
| | | or differentiation. |
| 369 | 393 | HT protein is a secreted glycoprotein with an EGF-like |
| | | domain. It functions as a modulator of cell growth, death or |
| | | differentiation. |
| 467 | 489 | Myb proto-oncogene (c-Myb), involved in transcription |
| | | regulation and activation of transcription |
| 471 | 493 | Chondroitin sulfotransferase, a member of the HNK-1 |
| | | sulfotransferase family. These molecules are involved in |
| | | the pathogenesis of arteriosclerosis, and proliferation of |
| | | arterial smooth muscle cells during development of |
| | | arteriosclerosis. |
| 472 | 494 | 36 kDa nucleolar protein HNP36, a novel growth factor |
| | | responsive gene expressed in the pituitary and parathyroid |
| | | glands |
| 475 | 497 | Zinc protease is a matrix metalloproteinase whose activity |
| | | is directed against components of the extracellular matrix |
| | | and play an important role in the growth, metastasis and |
| | | angiogenesis of tumors. |
| 476 | 498 | Diapophytoene dehydrogenase crtn-like molecule. This |
| | | molecule is similar to the diapophytoene dehydrogenase crt |
| | | molecule in a major photosynthesis gene cluster from the |
| | | bacterium Heliobacillus mobilis |
| 477 | 499 | Protocadherin 3 family member, involved in cell to cell |
| | | interactions. |
| 478 | 500 | Integrins are Type I membrane proteins that function as |
| | | laminin and collagen receptors and play a role in cell |
| | | adhesion. |
| 479 | 501 | Integrin family member. Integrins are Type I membrane |
| | | proteins that function as laminin and collagen receptors and |
| | | play a role in cell adhesion. |
| 480 | 502 | Similar to secreted HT Protein, a secreted glycoprotein with |
| | | an EGF-like domain. It functions as a modulator of cell |
| | | growth, death or differentiation |
| 481 | 503 | Agrin family member: Agrin is produced by motoneurons |
| | | and induces the aggregation of nicotinic acetylcholine |
| | | receptors. |
| 482 | 504 | Macrophage Scavenger Receptors bind to a variety of |
| | | polyanionic ligands and display complex binding |
| | | characteristics. They have been implicated in various |
| | | macrophage-associated processes, including atherosclerosis. |
| 483 | 505 | Similar to GARP, a member of the family of leucine-rich |
| | | repeat-containing proteins involved in platelet-endothelium |
| | | interactions. |
| 484 | 506 | Epidermal growth factor family proteins which stimulate |
| | | growth and mobility of keratinocytes and epithelial cells. |
| | | EGF is involved in wound healing. It also inhibits gastric |
| | | acid secretion. |
| 485 | 507 | Colony stimulating growth factor family. |
| 486 | 508 | Cytokine receptors |
| 487 | 509 | IL17 Receptor to Interleukin 17 (IL17), a T cell derived |
| | | cytokine that may play a role in initiation or maintenance of |
| | | the inflammatory response. |
| 438 | 458 | MEGF6, a protein containing multiple EGF-like-domains. |
| 439 | 459 | Protein kinase family member involved in signal |
| | | transduction. |
| 454 | | Peroxisomal calcium-dependent solute carrier, a new |
| | | member of the mitochondrial transporter superfamily. |
| 511 | 513 | Serine/threonine kinase NEK1 is a NIMA-related protein |
| | | kinase that phosphorylates serines and threonines, but also |
| | | possesses tyrosine kinase activity. NEK1 has been |
| | | implicated in the control of meiosis and belongs to the |
| | | NIMA kinase subfamily. |
| 514 | 624 | 626 Homologue isolated from rat dermal papilla of integrin |
| | | alpha-11/beta-1 that is involved in muscle development and |
| | | maintaining integrity of adult muscle and other adult |
| | | tissues. Integrin alpha-11/beta-1 is a receptor for collagen |
| | | and belongs to the integrin alpha chain family. |
| 516 | 625 | This is a secreted molecule isolated from rat dermal papillae |
| | | with a signal peptide at the N-terminus (amino acid residues |
| | | 1 to 21; nucleotides 42 to 104). |
| 517 | 626 | Homologue isolated from a rat dermal papilla library of |
| | | OASIS (old astrocyte specifically-induced substance) and |
| | | that plays a role in regulation of the response of astrocytes |
| | | to inflammation and trauma of the central nervous system |
| | | (CNS) during gliosis. The OASIS gene encodes a putative |
| | | transcription factor belonging to the cyclic AMP responsive |
| | | element binding protein/activating transcription factor |
| | | (CREB/ATE) gene family (Honma et al., Brain Res. Mol. |
| | | Brain Res. 69: 93-103, 1999). |
| 519 | 628 | This is a secreted molecule isolated from rat dermal papillae |
| | | with a signal peptide at the N-terminus (amino acid residues |
| | | 1 to 24; nucleotides 50 to 121). |
| 520 | 630 | This is a secreted molecule isolated from rat dermal papillae |
| | | with a signal peptide at the N-terminus (amino acid residues |
| | | 1 to 35; nucleotides 67 to 171). |
| 523 | 633 | This is a secreted molecule isolated from rat dermal papillae |
| | | with a signal peptide at the N-terminus (amino acid residues |
| | | 1 to 17; nucleotides 3 to 53). |
| 524 | 634 | This is a secreted molecule isolated from rat dermal papillae |
| | | with a signal peptide at the N-terminus (amino acid residues |
| | | 1 to 20; nucleotides 13 to 72). |
| 525, | 635, | Homologue isolated from a rat dermal papilla library of |
| 534 | 644 | leucyl-specific aminopeptidase, PILS-AP and that plays role |
| | | in many physiological processes as a substrate-specific |
| | | peptidase. PILS is a new member of the M1 famile of Zn- |
| | | dependent aminopeptidases that comprises members of |
| | | closely related enzymes which are known to be involved in |
| | | a variety of physiologically important processes. |
| 526 | 636 | This is a secreted molecule isolated from rat dermal papillae |
| | | with a signal peptide at the N-terminus (amino acid residues |
| | | 1 to 26; nucleotides 114 to 191). |
| 527 | 637 | This is a secreted molecule isolated from rat dermal papillae |
| | | with a signal peptide at the N-terminus (amino acid residues |
| | | 1 to 26; nucleotides 23 to 100). |
| 529 | 639 | This is a secreted molecule isolated from rat dermal papillae |
| | | with a signal peptide at the N-terminus (amino acid residues |
| | | 1 to 17; nucleotides 37 to 87). |
| 530 | 640 | This is a homologue isolated from a rat dermal papilla |
| | | library of a maturase that is involved in RNA splicing. |
| 531 | 641 | This is a secreted molecule isolated from rat dermal papillae |
| | | with a signal peptide at the N-terminus (amino acid residues |
| | | 1 to 17; nucleotides 180 to 230). |
| 532 | 642 | This is a secreted molecule isolated from rat dermal papillae |
| | | with a signal peptide at the N-terminus (amino acid residues |
| | | 1 to 32; nucleotides 245 to 340). |
| 535 | 645 | This is a secreted molecule isolated from rat dermal papillae |
| | | with a signal peptide at the N-terminus (amino acid residues |
| | | 1 to 25; nucleotides 188 to 333). |
| 536 | 646 | This is a secreted molecule isolated from rat dermal papillae |
| | | with a signal peptide at the N-terminus (amino acid residues |
| | | 1 to 21; nucleotides 185 to 247). |
| 537 | 647 | This is a secreted molecule isolated from rat dermal papillae |
| | | with a signal peptide at the N-terminus (amino acid residues |
| | | 1 to 24; nucleotides 129 to 200). |
| 541 | 651 | This is a homologue isolated from a rat dermal papilla |
| | | library of a hepatoma-derived growth factor (HDGF) that is |
| | | involved in stimulation of cell proliferation. |
| 542 | 652 | This is a receptor-like molecule isolated from rat dermal |
| | | papillae with two transmembrane domains (amino acid |
| | | residues 20 to 40 and 58 to 78. |
| 545 | 655 | This is a homologue isolated from a rat dermal papilla |
| | | library of Link protein (LP) and that is involved in bone |
| | | formation. LP plays an essential role in endochondral bone |
| | | formation by stabilizing the supramolecular assemblies of |
| | | aggrecan and hyaluronan (Deak et al., Cytogenet. Cell |
| | | Genet. 87: 75-79, 1999). |
| 548 | 658 | This is a homologue isolated from a rat dermal papilla |
| | | library of thrombospondin (TSP). It is a secreted protein |
| | | with a signal peptide in amino acid residues 1 to 18 |
| | | (nucleotides 210 to 263). TSP is an extracellular matrix |
| | | glycoprotein whose expression has been associated with a |
| | | variety of cellular processes including growth and |
| | | embryogenesis (Laherty et al., J. Biol. Chem. 267: 3, 274- |
| | | 3, 281, 1992). |
| 553 | 662 | This is a receptor-like molecule isolated from rat dermal |
| | | papillae with a transmembrane domain (amino acid residues |
| | | 434 to 454. |
| 554 | 663 | This is a receptor-like molecule isolated from rat dermal |
| | | papillae with a transmembrane domain (amino acid residues |
| | | 546 to 566. |
| 555 | 664 | This is a homologue isolated from a rat dermal papilla |
| | | library of B7-like mouse GL50 (mGL50). It is a receptor- |
| | | like molecule with a signal peptide in residues 1 to 24 |
| | | (nucleotides 149 to 220) and a transmembrane domain in |
| | | amino acid residues 262 to 282. GL50 is a specific ligand |
| | | for the ICOS receptor and this interaction functions in |
| | | lymphocyte costimulation (Ling et al., J. Immunol. |
| | | 164: 1,653-1,657, 2000). |
| 557, | 666, | These molecules are differentially expressed in stem cells |
| 558, | 667, | but not in mature keratinocytes and are involved in |
| 561- | 670- | developmental processes. They may be employed for |
| 572 | 678 | diagnosis of tumors with an immature phenotype. |
| 559 | 668 | This is a homologue isolated from a mouse stem cell library |
| | | of ABSENT IN MELANOMA 1 protein AIM1 and that can |
| | | be used for diagnosis of tumours with an immature |
| | | phenotype. AIM1 is a novel gene whose expression is |
| | | associated with the experimental reversal of tumorigenicity |
| | | of human malignant melanoma and belongs to the |
| | | betagamma-crystallin superfamily (Ray et al., Proc. Natl. |
| | | Acad. Sci. USA 94: 3,229-3,234, 1997) |
| 560 | 669 | Homologue isolated from a mouse stem cell library of |
| | | endothelin-convertin enzyme 2 (ECE-2) and that can be |
| | | used for diagnosis of tumours with an immature phenotype. |
| | | Endothelins (ET) are a family of potent vasoactive peptides |
| | | that are produced from biologically inactive intermediates, |
| | | termed big endothelins, via a proteolytic processing at |
| | | Trp21-Val/Ile22. ECE-2, that produces mature ET-1 from |
| | | big ET-1 both in vitro and in transfected cells. ECE-2 acts |
| | | as an intracellular enzyme responsible for the conversion of |
| | | endogenously synthesized big ET-1 at the trans-Golgi |
| | | network, where the vesicular fluid is acidified (Emoto and |
| | | Yanagisawa, J. Biol. Chem. 270: 15,262-15,268, 1995). |
| 573 | 679 | Mouse homologue of EGF-like molecule containing mucin- |
| | | like hormone receptor 2 (EMR2). The isolated molecule |
| | | contains three transmembrane regions: amino acid residues |
| | | 20 to 40, 66 to 86 and 92 to 112. The epidermal growth |
| | | factor (EGF)-TM7 proteins [EMR1 and EMR2, F4/80, and |
| | | CD97] constitute a recently defined class B GPCR |
| | | subfamily and are predominantly expressed on leukocytes. |
| | | These molecules possess N-terminal EGF-like domains |
| | | coupled to a seven-span transmembrane (7TM) moiety via a |
| | | mucin-like spacer domain (Lin et al., Genomics |
| | | 67: 188-200, 2000). |
| 574 | 680 | This is a murine secreted molecule with a signal peptide at |
| | | the N-terminus (amino acid residues 1 to 17; nucleotides |
| | | 238 to 288). |
| 575 | 681 | Mouse homologue of a glucocortocoid-inducible protein |
| | | GIS5 with a signal peptide at the N-terminus (amino acid |
| | | residues 1 to 17; nucleotides 56-106). |
| 576 | 682 | This is a murine surface receptor-like molecule with a |
| | | signal peptide at the N-terminus (amino acid residues 1 to |
| | | 17; nucleotides 1179 to 199) and a transmembrane domain |
| | | (amino acid residues 179 to 199). |
| 577 | 683 | This is a murine secreted molecule with a signal peptide at |
| | | the N-terminus (amino acid residues 1 to 16; nucleotides 55 |
| | | to 102). |
| 578 | 684 | This is a murine secreted molecule with a signal peptide at |
| | | the N-terminus (amino acid residues 1 to 22; nucleotides 12 |
| | | to 77). |
| 579 | 685 | This is a murine secreted molecule with a signal peptide at |
| | | the N-terminus (amino acid residues 1 to 17; nucleotides 82 |
| | | to 132). |
| 580 | 686 | This is a murine secreted molecule with a signal peptide at |
| | | the N-terminus (amino acid residues 1 to 20; nucleotides 20 |
| | | to 79). |
| 581 | 687 | This is a murine receptor-like molecule with |
| | | transmembrane domains at amino acid residues 50 to 70; 84 |
| | | to 104; 116 to 136 and 179 to 198. |
| 585 | 691 | This is a murine secreted molecule with a signal peptide at |
| | | the N-terminus (amino acid residues 1 to 20; nucleotides |
| | | 260 to 319). |
| 586 | 695 | This is a murine secreted molecule with a signal peptide at |
| | | the N-terminus (amino acid residues 1 to 22; nucleotides |
| | | 295 to 360). |
| 587 | 693 | This is a mouse homologue of serotransferrin, also known |
| | | as siderophilin or beta-1-metal binding globulin) and that is |
| | | involved in iron transport. This homologue is a secreted |
| | | molecule with a signal peptide at the N-terminus (amino |
| | | acid residues 1 to 19; nucleotides 43 to 99). Transferrins are |
| | | iron binding transport proteins which can bind two atoms of |
| | | ferric iron in association with the binding of an anion, |
| | | usually bicarbonate. It is responsible for the transport of |
| | | iron from sites of absorption and heme degradation to those |
| | | of storage and utilization. Serum transferrin may also have |
| | | a further role in stimulating cell proliferation. Transferrin |
| | | belongs to the transferrin family. |
| 589 | 695 | This is a murine secreted molecule with a signal peptide at |
| | | the N-terminus (amino acid residues 1 to 25; nucleotides 1 |
| | | to 75). |
| 592 | 697 | This is a murine receptor-like molecule with a |
| | | transmembrane domain in amino acid residues 52 to 72. |
| 593 | 698 | Mouse homologue of channel inducing factor (CHIF) that |
| | | plays a role in ion transport. The mouse homologue has a |
| | | signal peptide at the N-terminus of the predicted |
| | | polypeptide (amino acid residues 1 to 20; nucleotides 102 to |
| | | 161) and a transmembrane domain (amino acid residues 38 |
| | | to 58). CHIF evokes a potassium channel activity (Attali et |
| | | al., Proc. Natl. Acad. Sci. USA 92: 6092-6096, 1995). |
| 595 | 700 | Homologue of hyaluronan receptor LYVE-1 that plays a |
| | | role in hyalyronan uptake. This mouse homologue has the |
| | | characteristic signal peptide and transmembrane domain of |
| | | a receptor. A signal peptide was identified in the isolated |
| | | molecule in amino acid residues 1 to 18 (nucleotides 62 to |
| | | 115) and the transmembrane domain in amino acid residues |
| | | 233 to 253. The extracellular matrix glycosaminoglycan |
| | | hyaluronan (HA) is an abundant component of skin and |
| | | mesenchymal tissues where it facilitates cell migration |
| | | during wound healing, inflammation, and embryonic |
| | | morphogenesis. Both during normal tissue homeostasis and |
| | | particularly after tissue injury, HA is mobilized from these |
| | | sites through lymphatic vessels to the lymph nodes where it |
| | | is degraded before entering the circulation for rapid uptake |
| | | by the liver. LYVE-1 is a receptor for HA on the lymph |
| | | vessel wall and plays a role in the transport of HA from |
| | | tissue to lymph (Banerji et al., J. Cell Biol. 144: 789- |
| | | 801, 1999). |
| 596 | 701 | This is a murine secreted molecule with a signal peptide at |
| | | the N-terminus (amino acid residues 1 to 21; nucleotides 7 |
| | | to 69). |
| 598 | 703 | Homologue of tumor-associated glycoprotein E4 (TAA1 or |
| | | TAGE4) that belongs to the immunoglobulin superfamily. |
| | | This molecule has a signal peptide at the N-terminus (amino |
| | | acid residues 1 to 24; nucleotides 71 to 142) and is |
| | | therefore a secreted protein. |
| 599 | 704 | Homologue of the LUNX protein, also known as |
| | | nasopharyngeal carcinoma-related protein, tracheal |
| | | epithelium enriched protein or plunc, that is expressed in |
| | | epithelial cells in the airways. It has a signal peptide at the |
| | | N-terminus (amino acid residues 1 to 19; nucleotides 39 to |
| | | 95). Expression of LUNX is restricted to the trachea, upper |
| | | airway, nasopharyngeal epithelium and salivary gland |
| | | (Bingle and Bingle, Biochim. Biophys. Acta 1493: 363- |
| | | 367, 2000). |
| 600 | 705 | This is a murine secreted molecule with a signal peptide at |
| | | the N-terminus (amino acid residues 1 to 23; nucleotides |
| | | 136 to 204. |
| 601 | 706 | Homologue of prenylcysteine lyase (EC 4.4.1.18) and that |
| | | is involved in degradation of prenylated proteins. It has a |
| | | signal peptide at the N-terminus (amino acid residues 1 to |
| | | 28; nucleotides 22 to 105). Prenylcysteine lyase is a specific |
| | | enzyme involved in the final step of prenylcysteine |
| | | metabolism in mammalian cells. The enzyme does not |
| | | require NADPH as cofactor for prenylcysteine degradation, |
| | | thus distinguishing it from cytochrome P450- and flavin- |
| | | containing monooxygenases that catalyze S-oxidation of |
| | | thioethers (Zhang et al., J. Biol. Chem. 274: 35802-35808, |
| | | 1999). |
| 605 | 710 | Homologue of endoplasmin, endoplasmic reticulum protein |
| | | 99 (ERp99), 94 kDa glucose-regulated protein (GRP94) and |
| | | polymorphic tumor rejection antigen 1 (gp96). The isolated |
| | | molecule has a signal peptide at the N-terminus (amino acid |
| | | residue 1 to 21; nucleotides 1867 to 206). ERp99 is an |
| | | abundant, conserved transmembrane glycoprotein of the |
| | | endoplasmic reticulum membrane and homologous to the |
| | | 90-kDa heat shock protein (hsp90) and the 94-kDa glucose |
| | | regulated protein (GRP94) (Mazzarella and Green, J. Biol. |
| | | Chem. 262: 8875-8883, 1987). |
| 606 | 711 | Homologue of PILRalpha, formerly known as inhibitory |
| | | receptor PIRIIalpha and that is involved in signal |
| | | transduction in various cellular processes. This molecule |
| | | contains a signal peptide at the N-terminal end (amino acid |
| | | residues 1-21 and nucleotides 47 to 139) and a |
| | | transmembrane domain at amino acid residues 191 to 211. |
| | | SHP-1-mediated dephosphorylation of protein tyrosine |
| | | residues is central to the regulation of several cell signaling |
| | | pathways. PILRalpha, a novel immunoreceptor tyrosine- |
| | | based inhibitory motif-bearing protein, recruits SHP-1 upon |
| | | tyrosine phosphorylation and is paired with the truncated |
| | | counterpart PILRbeta (Mousseau et al., J. Biol. Chem. |
| | | 275: 4467-4474, 2000). |
| 607 | 712 | This is a murine secreted molecule with a signal peptide at |
| | | the N-terminus (amino acid residues 1 to 18; nucleotides 38 |
| | | to 91. |
| 609 | 714 | Homologue of retinal short-chain dehydrogenase/reductase |
| | | retSDR2 that plays a role on retinal metabolism. It has a |
| | | signal peptide at the N-terminus at amino acid residues 1- |
| | | 29 (nucleotides 302 to 388). Retinol dehydrogenases |
| | | (RDH) catalyze the reduction of all-trans-retinal to all-trans- |
| | | retinol within the photoreceptor outer segment in the |
| | | regeneration of bleached visual pigments (Haeseleer et al., |
| | | J. Biol. Chem. 273: 21790-21799, 1998) |
| 612 | 717 | This is a murine secreted molecule with a signal peptide at |
| | | the N-terminus (amino acid residues 1 to 22; nucleotides 6 |
| | | to 71. |
| 613 | 718 | This is a murine secreted molecule with a signal peptide at |
| | | the N-terminus (amino acid residues 1 to 25; nucleotides |
| | | 210 to 284. |
| 615 | 720 | This is a murine secreted molecule with a signal peptide at |
| | | the N-terminus (amino acid residues 1 to 16; nucleotides 70 |
| | | to 117. |
| 616 | 721 | This is a murine secreted molecule with a signal peptide at |
| | | the N-terminus (amino acid residues 1 to 18; nucleotides 1 |
| | | to 54. |

The location of open reading frames (ORFs) within certain of the inventive cDNA sequences are shown in Table 3, below.

TABLE 3
LOCATION OF OPEN READING FRAMES

| SEQ ID NO | | SEQ ID NO |
|---|---|---|
| Polynucleotide | ORF | Polypeptide |
| 514 | 1-2,067 | 624 |
| 515 | 2-730 | 625 |
| 516 | 42-1,772 | 626 |
| 517 | 1-681 | 627 |
| 518 | 170-416 | 628 |
| 519 | 50-770 | 629 |
| 520 | 67-708 | 630 |
| 521 | 110-613 | 631 |
| 522 | 41-457 | 632 |
| 523 | 3-230 | 633 |
| 524 | 13-573 | 634 |
| 525 | 64-2,856 | 635 |
| 526 | 114-599 | 636 |
| 527 | 23-520 | 637 |
| 528 | 953-1,138 | 638 |
| 529 | 37-687 | 639 |
| 530 | 145-366 | 640 |
| 531 | 180-1,508 | 643 |
| 532 | 245-442 | 642 |
| 533 | 125-595 | 643 |
| 534 | 64-2,856 | 644 |
| 535 | 188-727 | 645 |
| 536 | 185-1,081 | 646 |
| 537 | 129-308 | 647 |
| 538 | 32-853 | 648 |
| 539 | 2-268 | 649 |
| 540 | 3-875 | 650 |
| 541 | 284-892 | 651 |
| 542 | 37-276 | 652 |
| 543 | 127-1,794 | 653 |
| 544 | 1-735 | 654 |
| 545 | 142-939 | 655 |
| 546 | 51-1,082 | 656 |
| 547 | 143-328 | 657 |
| 548 | 210-3,728 | 658 |
| 549 | 26-1,354 | 659 |
| 551 | 1,236-1,892 | 660 |
| 552 | 853-1,178 | 661 |
| 553 | 54-1,356 | 662 |
| 554 | 637-2,244 | 663 |
| 555 | 149-1,072 | 664 |
| 556 | 18-449 | 665 |
| 557 | 275-1,171 | 666 |
| 558 | 453-1,133 | 667 |
| 559 | 104-2,449 | 668 |
| 560 | 463-687 | 669 |
| 562 | 1-1,107 | 670 |
| 563 | 2-883 | 671 |
| 564 | 188-2,902 | 672 |
| 565 | 3-524 | 673 |
| 567 | 2,584-3,996 | 674 |
| 569 | 1-960 | 675 |
| 570 | 315-599 | 676 |
| 571 | 1-414 | 677 |
| 572 | 806-1,912 | 678 |
| 573 | 120-752 | 679 |
| 574 | 2381, 359 | 680 |
| 575 | 56-1,456 | 681 |
| 576 | 13-645 | 682 |
| 577 | 55-1,323 | 683 |
| 578 | 12-698 | 684 |
| 579 | 82-810 | 685 |
| 580 | 20-586 | 686 |
| 581 | 65-808 | 687 |
| 582 | 369-761 | 688 |
| 583 | 1-769 | 689 |
| 584 | 164-1,321 | 690 |
| 585 | 260-1,489 | 691 |
| 586 | 295-1,131 | 692 |
| 587 | 43-2,136 | 693 |
| 588 | 1-1,203 | 694 |
| 589 | 1-525 | 695 |
| 591 | 1-584 | 696 |
| 592 | 1-522 | 697 |
| 593 | 102-368 | 698 |
| 594 | 1-517 | 699 |
| 595 | 62-1,018 | 700 |
| 596 | 7-282 | 701 |
| 597 | 1-736 | 702 |
| 598 | 71-1,297 | 703 |
| 599 | 39-875 | 704 |
| 600 | 136-930 | 705 |
| 601 | 22-1,539 | 706 |
| 602 | 69-521 | 707 |
| 603 | 104-448 | 708 |
| 604 | 1-399 | 709 |
| 605 | 3,068-5,476 | 710 |
| 606 | 47-721 | 711 |
| 607 | 38-439 | 712 |
| 608 | 1-1,656 | 713 |
| 609 | 302-1,327 | 714 |
| 610 | 845-1,447 | 715 |
| 611 | 975-1,375 | 716 |
| 612 | 6-272 | 717 |
| 613 | 210-464 | 718 |
| 614 | 462-869 | 719 |
| 615 | 70-459 | 720 |
| 616 | 1-1,107 | 721 |
| 617 | 1-349 | 722 |
| 618 | 93-528 | 723 |
| 621 | 380-1,033 | 724 |
| 622 | 43-2,115 | 725 |

The cDNA sequences of SEQ ID NO: 514, 515, 516, 557, 558, 559, 560, 561, 567, 568, 619 and 621 are extended sequences of SEQ ID NO: 479, 480, 353, 91, 108, 82, 92, 81, 105, 90, 362 and 360, respectively. SEQ ID NO: 516, 520, 521, 523, 525, 526, 529, 534-536, 541-543, 546, 548, 549, 557, 574, 575, 577-581, 584-587, 589, 593, 595, 596, 598-601, 605, 607, 609, 610, 614, 616 and 622 represent full-length cDNA sequences.

The polynucleotide sequences of SEQ ID NOS: 77-117, 265-267, 404-405 and 557-611 are differentially expressed in either keratinocyte stem cells (KSCL) or in transit amplified cells (TRAM) on the basis of the number of times these sequences exclusively appear in either one of the above two libraries; more than 9 times in one and none in the other (Audic S. and Claverie J-M, Genome Research, 7:986-995, 1997). The sequences of SEQ ID NOS: 77-89, 265-267 and 365-369 were determined to have less than 75% identity to sequences in the EMBL database using the computer algorithm FASTA or BLASTN, as described above. The polypeptide sequences encoded by the cDNA sequences of SEQ ID NO: 77-117, 265-267, 404-405 and 557-611 are provided in SEQ ID NOS: 666-718. The amino acid sequences of SEQ ID NOS: 666, 668, 669, 671-673, 675, 676, 679, 682, 683, 685, 688, 690, 691, 693, 694, 702, 703, 706-708, 710, 711, 713 and 714 show less than 75% identity to sequences in the SwissProt database.

The polypeptides encoded by these polynucleotide sequences have utility as markers for identification and isolation of these cell types, and antibodies against these proteins may be usefully employed in the isolation and enrichment of these cells from complex mixtures of cells. Isolated polynucleotides and their corresponding proteins exclusive to the stem cell population can be used as drug targets to cause alterations in regulation of growth and differentiation of skin cells, or in gene targeting to transport specific therapeutic molecules to skin stem cells.

### EXAMPLE 3

### Isolation and Characterization of the Human Homolog of muTR1

The human homolog of muTR1 (SEQ ID NO: 68), obtained as described above in Example 1, was isolated by screening 50,000 pfu's of an oligo dT primed HeLa cell cDNA library. Plaque lifts, hybridization, and screening were performed using standard molecular biology techniques (Sambrook, J, Fritsch, E F and Maniatis, T, eds., Molecular Cloning: A Laboratory Manual, 2nd ed., Cold Spring Harbor Laboratory Press, Cold Spring Harbor, N.Y., 1989). The determined cDNA sequence of the isolated human homolog (huTR1) is provided in SEQ ID NO: 118, with the corresponding polypeptide sequence being provided in SEQ ID NO: 196. The library was screened using an [α³²P]-dCTP labeled double stranded cDNA probe corresponding to nucleotides 1 to 459 of the coding region within SEQ ID NO: 118.

The polypeptide sequence of huTR1 has regions similar to Transforming Growth Factor-alpha, indicating that this protein functions as an epidermal growth factor (EGF). EGF family members exist in a functional form as small peptides. Alignment of the functional peptides of the EGF family with SEQ ID NO: 196 revealed that an internal segment of SEQ ID NO: 196 (amino acids 54-104; SEQ ID NO: 343) shows greater than 40% identity to the active peptides of EGF, TGF-alpha and Epiregulin. The active peptides of the EGF family are sufficient for activity and contain several conserved residues critical for the maintenance of this activity. These residues are retained in huTR1. The inventive EGF-like protein will serve to stimulate keratinocyte growth and motility, and to inhibit the growth of epithelial-derived cancer cells. This novel gene and its encoded protein may thus be used as agents for the healing of wounds and regulators of epithelial-derived cancers.

Analysis of RNA Transcripts by Northern Blotting

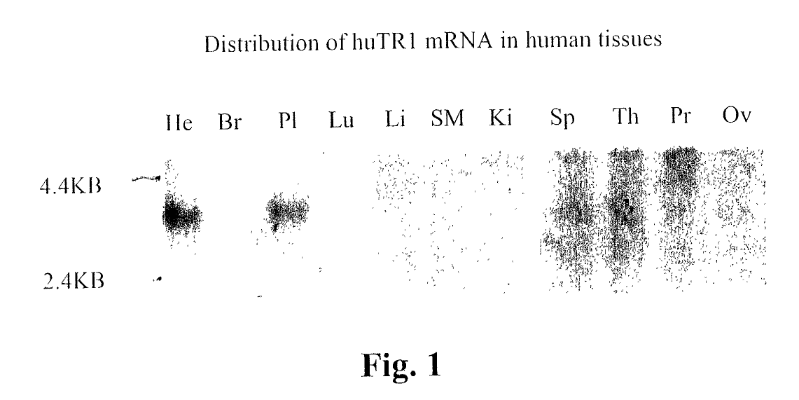

Northern analysis to determine the size and distribution of mRNA for huTR1 was performed by probing human tissue mRNA blots (Clontech) with a probe comprising nucleotides 93-673 of SEQ ID NO: 118, radioactively labeled with [α³²P]-dCTP. Prehybridization, hybridization, washing and probe labeling were performed as described in Sambrook, et al., Ibid. mRNA for huTR1 was 3.5-4 kb in size and was observed to be most abundant in heart and placenta, with expression at lower levels being observed in spleen, thymus, prostate and ovary (FIG. 1).

The high abundance of mRNA for huTR1 in the heart and placenta indicates a role for huTR1 in the formation or maintenance of blood vessels, as heart and placental tissues have an increased abundance of blood vessels, and therefore endothelial cells, compared to other tissues in the body. This, in turn, demonstrates a role for huTR1 in angiogenesis and vascularization of tumors. This is supported by the ability of Transforming Growth Factor-alpha and EGF to induce de novo development of blood vessels (Schreiber, et al., Science 232:1250-1253, 1986) and stimulate DNA synthesis in endothelial cells (Schreiber, et al., Science 232:1250-1253, 1986), and their over-expression in a variety of human tumors.

Purification of muTR1 and huTR1

Polynucleotides 177-329 of muTR1 (SEQ ID NO: 268), encoding amino acids 53-103 of muTR1 (SEQ ID NO: 342), and polynucleotides 208-360 of huTR1 (SEQ ID NO: 269), encoding amino acids 54-104 of huTR1 (SEQ ID NO: 343), were cloned into the bacterial expression vector pProEX HT (BRL Life Technologies), which contains a bacterial leader sequence and N-terminal 6×Histidine tag. These constructs were transformed into competent XL1-Blue E. coli as described in Sambrook et al., Ibid.

Starter cultures of these recombinant XL1-Blue E. coli were grown overnight at 37° C. in Terrific broth containing 100 μg/ml ampicillin. This culture was spun down and used to inoculate 500 ml culture of Terrific broth containing 100 μg/ml ampicillin. Cultures were grown until the OD₅₉₅ of the cells was between 0.4 and 0.8, whereupon IPTG was added to 1 mM. Cells were induced overnight and bacteria were harvested by centrifugation.

Both the truncated polypeptide of muTR1 (SEQ ID NO: 342; referred to as muTR1a) and that of huTR1 (SEQ ID NO: 343; referred to as huTR1a) were expressed in insoluble inclusion bodies. In order to purify the polypeptides muTR1a and huTR1a, bacterial cell pellets were re-suspended in lysis buffer (20 mM Tris-HCl pH 8.0, 10 mM beta mercaptoethanol, 1 mM PMSF). To the lysed cells, 1% NP40 was added and the mix incubated on ice for 10 minutes. Lysates were further disrupted by sonication on ice at 95W for 4×15 seconds and then centrifuged for 15 minutes at 14,000 rpm to pellet the inclusion bodies.

The resulting pellet was re-suspended in lysis buffer containing 0.5% w/v CHAPS and sonicated on ice for 5-10 seconds. This mix was stored on ice for 1 hour, centrifuged at 14,000 rpm for 15 minutes at 4° C. and the supernatant discarded. The pellet was once more re-suspended in lysis buffer containing 0.5% w/v CHAPS, sonicated, centrifuged and the supernatant removed as before. The pellet was re-suspended in solubilizing buffer (6 M Guanidine HCl, 0.5 M NaCl, 20 mM Tris HCl, pH 8.0), sonicated at 95 W for 4×15 seconds and then centrifuged for 20 minutes at 14,000 rpm and 4° C. to remove debris. The supernatant was stored at 4° C. until use.

Polypeptides muTR1a and huTR1a were purified by virtue of the N-terminal 6×Histidine tag contained within the bacterial leader sequence, using a Nickel-Chelating Sepharose column (Amersham Pharmacia, Uppsala, Sweden) and following the manufacturer's recommended protocol. In order to refold the proteins once purified, the protein solution was added to 5× its volume of refolding buffer (1 mM EDTA, 1.25 mM reduced glutathione, 0.25 mM oxidized glutathione, 20 mM Tris-HCl, pH 8.0) over a period of 1 hour at 4° C. The refolding buffer was stirred rapidly during this time, and stirring continued at 4° C. overnight. The refolded proteins were then concentrated by ultrafiltration using standard protocols.

Biological Activities of Polypeptides muTR1a and huTR1a

muTR1 and huTR1 are novel members of the EGF family, which includes EGF, TGFα, epiregulin and others. These growth factors are known to act as ligands for the EGF receptor. The pathway of EGF receptor activation is well documented. Upon binding of a ligand to the EGF receptor, a cascade of events follows, including the phosphorylation of proteins known as MAP kinases. The phosphorylation of MAP kinase can thus be used as a marker of EGF receptor activation. Monoclonal antibodies exist which recognize the phosphorylated forms of 2 MAP kinase proteins—ERK1 and ERK2.

In order to examine whether purified polypeptides of muTR1a and huTR1a act as a ligand for the EGF receptor, cells from the human epidermal carcinoma cell line A431 (American Type Culture Collection, No. CRL-1555, Manassas, Va.) were seeded into 6 well plates, serum starved for 24 hours, and then stimulated with purified muTR1a or huTR1a for 5 minutes in serum free conditions. As a positive control, cells were stimulated in the same way with 10 to 100 ng/ml TGF-alpha or EGF. As a negative control, cells were stimulated with PBS containing varying amounts of LPS. Cells were immediately lysed and protein concentration of the lysates estimated by Bradford assay. 15 μg of protein from each sample was loaded onto 12% SDS-PAGE gels. The proteins were then transferred to PVDF membrane using standard techniques.

For Western blotting, membranes were incubated in blocking buffer (10 mM Tris-HCl, pH 7.6, 100 mM NaCl, 0.1% Tween-20, 5% non-fat milk) for 1 hour at room temperature. Rabbit anti-Active MAP kinase pAb (Promega, Madison, Wis.) was added to 50 ng/ml in blocking buffer and incubated overnight at 4° C. Membranes were washed for 30 mins in blocking buffer minus non-fat milk before being incubated with anti rabbit IgG-HRP antibody, at a 1:3500 dilution in blocking buffer, for 1 hour at room temperature. Membranes were washed for 30 minutes in blocking buffer minus non-fat milk, then once for 5 minutes in blocking buffer minus non-fat milk and 0.1% Tween-20. Membranes were then exposed to ECL reagents for 2 min, and then autoradiographed for 5 to 30 min.

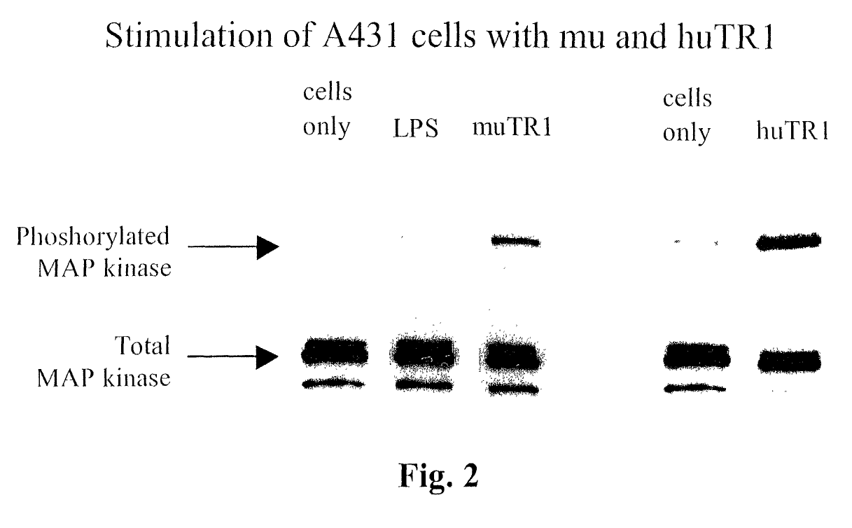

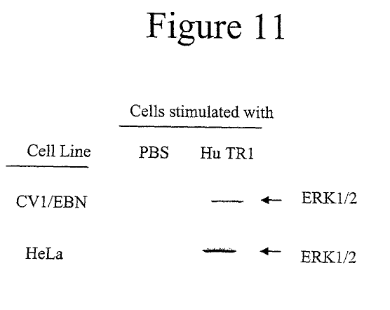

As shown in FIG. 2, both muTR1a and huTR1a were found to induce the phosphorylation of ERK1 and ERK2 over background levels, indicating that muTR1 and huTR1 act as ligands for a cell surface receptor that activates the MAP kinase signaling pathway, possibly the EGF receptor. As shown in FIG. 11, huTR1a was also demonstrated to induce the phosphorylation of ERK1 and ERK2 in CV1/EBNA kidney epithelial cells in culture, as compared with the negative control. These assays were conducted as described above. This indicates that huTR1a acts as a ligand for a cell surface receptor that activates the MAP kinase signaling pathway, possibly the EGF receptor in HeLa and CV1/EBNA cells.

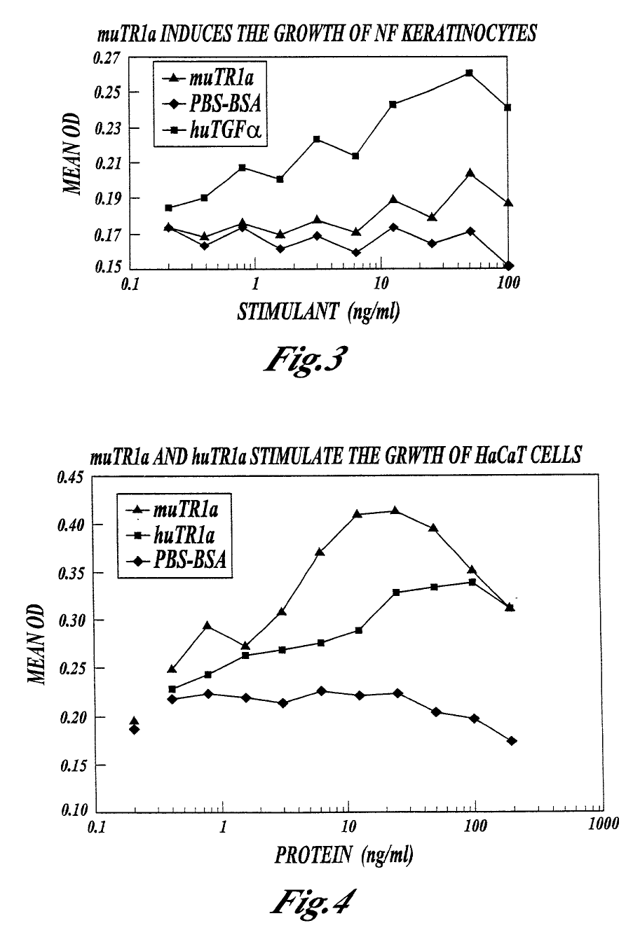

The ability of muTR1a to stimulate the growth of neonatal foreskin (NF) keratinocytes was determined as follows. NF keratinocytes derived from surgical discards were cultured in KSFM (BRL Life Technologies) supplemented with bovine pituatary extract (BPE) and epidermal growth factor (EGF). The assay was performed in 96 well flat-bottomed plates in 0.1 ml unsupplemented KSFM. MuTR1a, human transforming growth factor alpha (huTGFα) or PBS-BSA was titrated into the plates and 1×10³ NF keratinocytes were added to each well. The plates were incubated for 5 days in an atmosphere of 5% CO₂ at 37° C. The degree of cell growth was determined by MTT dye reduction as described previously (J. Imm. Meth. 93:157-165, 1986). As shown in FIG. 3, both muTR1a and the positive control human TGFα stimulated the growth of NF keratinocytes, whereas the negative control, PBS-BSA, did not.

The ability of muTR1a and huTR1a to stimulate the growth of a transformed human keratinocyte cell line, HaCaT, was determined as follows. The assay was performed in 96 well flat-bottomed plates in 0.1 ml DMEM (BRL Life Technologies) supplemented with 0.2% FCS. MuTR1a, huTR1a and PBS-BSA were titrated into the plates and 1×10³ HaCaT cells were added to each well. The plates were incubated for 5 days in an atmosphere containing 10% CO₂ at 37° C. The degree of cell growth was determined by MTT dye reduction as described previously (J. Imm. Meth. 93:157-165, 1986). As shown in FIG. 4, both muTR1a and huTR1a stimulated the growth of HaCaT cells, whereas the negative control PBS-BSA did not.

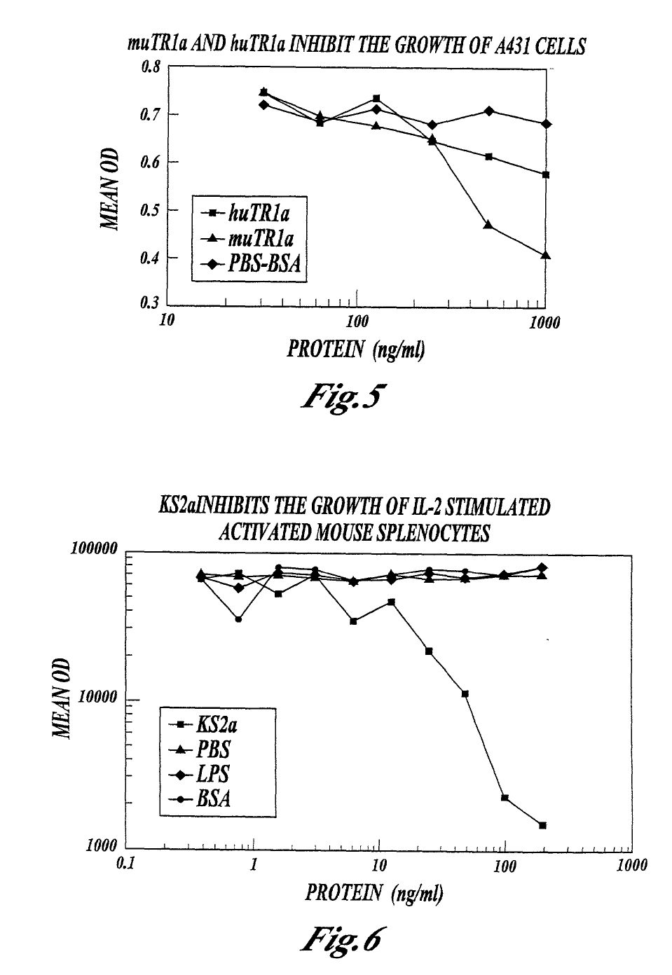

The ability of muTR1a and huTR1a to inhibit the growth of A431 cells was determined as follows. Polypeptides muTR1a (SEQ ID NO: 342) and huTR1a (SEQ ID NO: 343) and PBS-BSA were titrated as described previously (J. Cell. Biol. 93:1-4, 1982), and cell death was determined using the MTT dye reduction as described previously (J. Imm. Meth. 93:157-165, 1986). Both muTR1a and huTR1a were found to inhibit the growth of A431 cells, whereas the negative control PBS-BSA did not (FIG. 5).

These results indicate that muTR1 and huTR1 stimulate keratinocyte growth and motility, inhibit the growth of epithelial-derived cancer cells, and play a role in angiogenesis and vascularization of tumors. This novel gene and its encoded protein may thus be developed as agents for the healing of wounds, angiogenesis and regulators of epithelial-derived cancers.

Upregulation of huTR1 and mRNA Expression

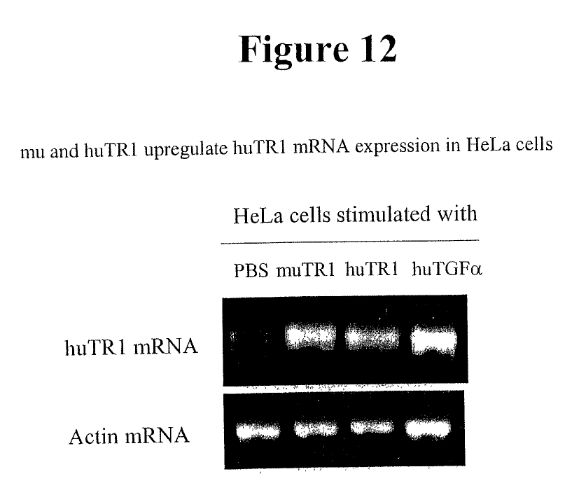

HeLa cells (human cervical adenocarcinoma) were seeded in 10 cm dishes at a concentration of 1×10⁶ cells per dish. After incubation overnight, media was removed and replaced with media containing 100 ng/ml of muTR1, huTR1, huTGFα, or PBS as a negative control. After 18 hours, media was removed and the cells lysed in 2 ml of TRIzol reagent (Gibco BRL Life Technologies, Gaithersburg, Md.). Total RNA was isolated according to the manufacturer's instructions. To identify mRNA levels of huTR1 from the cDNA samples, 1 μl of cDNA was used in a standard PCR reaction. After cycling for 30 cycles, 5 μl of each PCR reaction was removed and separated on a 1.5% agarose gel. Bands were visualized by ethidium bromide staining. As can be seen from FIG. 12, both mouse and human TR1 up-regulate the mRNA levels of huTR1 as compared with cells stimulated with the negative control of PBS. Furthermore, TGFα can also up-regulate the mRNA levels of huTR1.

These results indicate that TR1 is able to sustain its own mRNA expression and subsequent protein expression, and thus is expected to be able to contribute to the progression of diseases such as psoriasis where high levels of cytokine expression are involved in the pathology of the disease. Furthermore, since TGFα can up-regulate the expression of huTR1, the up-regulation of TR1 mRNA may be critical to the mode of action of TGFα.

Serum Response Element Reporter Gene Assay

The serum response element (SRE) is a promoter element required for the regulation of many cellular immediate-early genes by growth. Studies have demonstrated that the activity of the SRE can be regulated by the MAP kinase signaling pathway. Two cell lines, PC12 (rat pheochromocytoma—neural tumor) and HaCaT (human transformed keratinocytes), containing eight SRE upstream of an SV40 promotor and luciferase reporter gene were developed in-house. 5×10³ cells were aliquoted per well of 96 well plate and grown for 24 hours in their respective media. HaCaT SRE cells were grown in 5% fetal bovine serum (FBS) in D-MEM supplemented with 2 mM L-glutamine (Sigma, St. Louis, Mo.), 1 mM sodium pyruvate (BRL Life Technologies), 0.77 mM L-asparagine (Sigma), 0.2 mM arginine (Sigma), 160 mM penicillin G (Sigma), 70 mM dihydrostreptomycin (Roche Molecular Biochemicals, Basel, Switzerland), and 0.5 mg/ml geneticin (BRL Life Technologies). PC12 SRE cells were grown in 5% fetal bovine serum in Ham F12 media supplemented with 0.4 mg/ml geneticin (BRL Life Technologies). Media was then changed to 0.1% FBS and incubated for a further 24 hours. Cells were then stimulated with a titration of TR1 from 1 μg/ml. A single dose of basic fibroblast growth factor at 100 ng/ml (R&D Systems, Minneappolis, Minn.) or epidermal growth factor at 10 ng/ml (BRL Life Technologies) was used as a positive control. Cells were incubated in the presence of muTR1 or positive control for 6 hours, washed twice in PBS and lysed with 40 μl of lysis buffer (Promega). 10 μl was transferred to a 96 well plate and 10 μl of luciferase substrate (Promega) added by direct injection into each well by a Victor² fluorimeter (Wallac), the plate was shaken and the luminescence for each well read at 3×1 sec Intervals. Fold induction of SRE was calculated using the following equation: Fold induction of SRE=Mean relative luminescence of agonist/Mean relative luminescence of negative control.

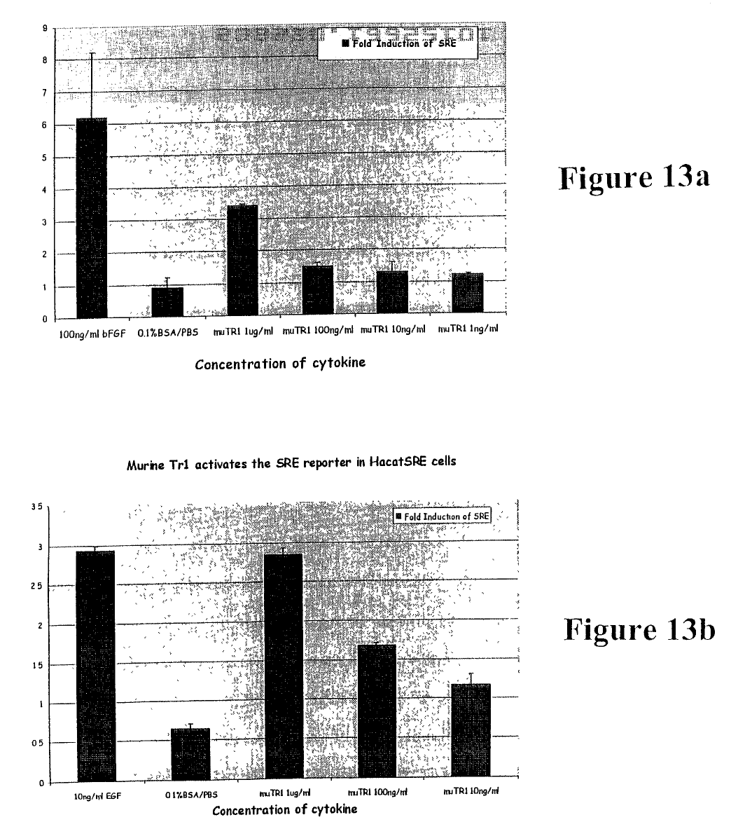

As shown in FIG. 13, muTR1 activated the SRE in both PC-12 (FIG. 13A) and HaCaT (FIG. 13B) cells. This indicates that HaCaT and PC-12 cells are able to respond to muTR1 protein and elicit a response. In the case of HaCaT cells, this is a growth response. In the case of PC-12 cells, this may be a growth, a growth inhibition, differentiation, or migration response. Thus, TR1 may be important in the development of neural cells or their differentiation into specific neural subsets. TR1 may also be important in the development and progression of neural tumors.

Inhibition by the EGF Receptor Assay

The HaCaT growth assay was conducted as previously described, with the following modifications. Concurrently with the addition of EGF and TR1 to the media, anti-EGF Receptor (EGFR) antibody (Promega, Madison, Wisconsin) or the negative control antibody, mouse IgG (PharMingen, San Diego, Calif.), were added at a concentration of 62.5 ng/ml.

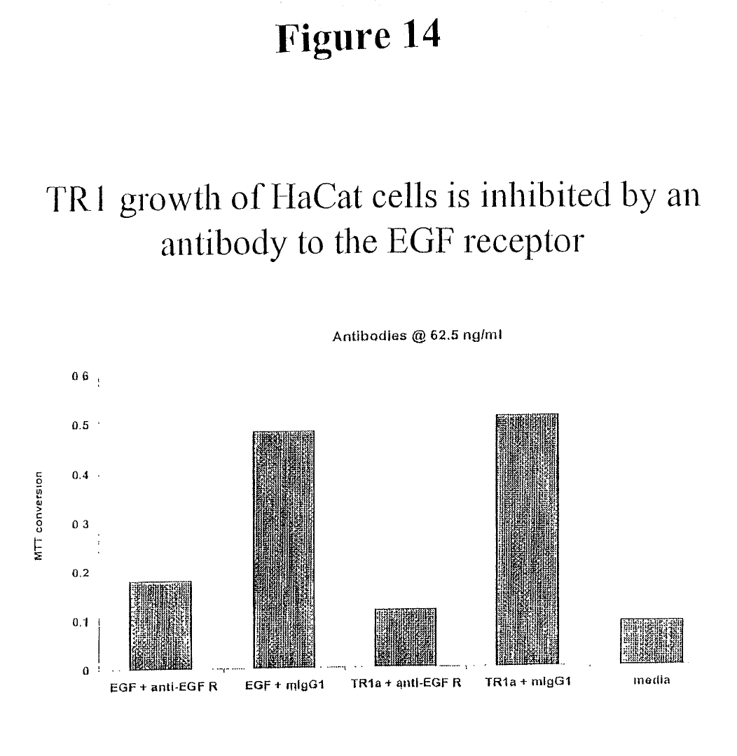

As seen in FIG. 14, an antibody which blocks the function of the EGFR inhibited the mitogenicity of TR1 on HaCaT cells. This indicates that the EGFR is crucial for transmission of the TR1 mitogenic signal on HaCaT cells. TR1 may bind directly to the EGF receptor. TR1 may also bind to any other members of the EGFR family (for example, ErbB-2, -3, and/or -4) that are capable of heterodimerizing with the EGFR.

Splice Variants of huTR1

A variant of huTR1 was isolated from the same library as huTR1, following the same protocols. The sequence referred to as huTR1-1 (also known as TR1δ) is a splice variant of huTR1 and consists of the ORF of huTR1 minus amino acids 15 to 44 and 87 to 137. These deletions have the effect of deleting part of the signal sequence and following amino terminal linker sequence, residues following the second cysteine residue of the EGF motif and the following transmembrane domain. However, cysteine residue 147 (huTR1 ORF numbering) may replace the deleted cysteine and thus the disulphide bridges are likely not affected. Therefore, huTR1-1 is an intracellular form of huTR1. It functions as an agonist or an antagonist to huTR1 or other EGF family members, including EGF and TGFα. The determined nucleotide sequence of huTR1-1, is given in SEQ ID NO: 412, with the corresponding amino acid sequence being provided in SEQ ID NO: 415.

Four additional splice variants of huTr1 were isolated by PCR on first strand cDNA made from RNA isolated from HeLa cells by standard protocols. These splice variants of huTR1 are referred to as TR1-2 (also known as TR1β), TR1-3 (also known as TR1γ), TR1ε and TR1φ.

TR1-2 consists of the ORF of huTR1 minus amino acids 95 to 137. This deletion has the effect of deleting the transmembrane domain. Therefore TR1-2 is a secreted form of huTR1 and binds with equal or greater affinity to the TR1 receptor as huTr1, since the EGF domain remains intact. It functions as an agonist or an antagonist to huTR1 or other EGF family members, including EGF and TGFα. The determined cDNA sequence of TR1-2 is given in SEQ ID NO: 410 and the corresponding amino acid sequence in SEQ ID NO: 413.

TR1-3 consists of the ORF of huTR1 minus amino acids 36 to 44 and amino acids 86 to 136. These deletions have the effect of deleting part of the amino terminal linker sequence, residues following the second cysteine of the EGF motif and the following transmembrane domain. However, cysteine residue 147 (huTR1 ORF numbering) may replace the deleted cysteine and thus the disulphide bridges are likely not affected. Therefore, TR1-3 is also a secreted form of huTR1 and functions as an agonist or an antagonist to huTR1 or other EGF family members, including EGF and TGFα. The determined cDNA sequence of TR1-3 is given in SEQ ID NO: 411 and the corresponding amino acid sequence is SEQ ID NO: 414.

TR1ε consists of the ORF of huTR1 minus amino acids 86 to 136. This deletion has the effect of deleting residues following the second cysteine of the EGF motif and the transmembrane domain. However, cysteine residue 147 (huTR1 ORF numbering) may replace the deleted cysteine and thus the disulphide bridges are likely not affected. Therefore, TR1ε is also a secreted form of huTR1 and functions as an agonist or an antagonist to huTR1 or other EGF family members, including EGF and TGFα. The determined cDNA sequence of TR1ε is given in SEQ ID NO: 371 and the corresponding polypeptide sequence in SEQ ID NO: 395.

TR1φ consists of the ORF of huTR1 minus amino acids 36 to 44 and amino acids 95 to 136. These deletions have the effect of deleting part of the amino terminal linker sequence and the transmembrane domain. Therefore TR1φ is a secreted form of huTR1 and binds with equal or greater affinity to the TR1 receptor as huTr1, since the EGF domain remains intact. It functions as an agonist or an antagonist to huTR1 or other EGF family members, including EGF and TGFα. The determined nucleotide sequence of TR1φ is given in SEQ ID NO: 416 and the corresponding polypeptide sequence in SEQ ID NO: 417.

Analysis of TR1 Expression using TR1-specific Antibodies

Polyclonal antibodies were generated to the active peptide of huTr1 by immunizing rabbits with huTR1 expressed in E. coli emulsified in complete Freunds adjuvant. The TR1-specific immune response was boosted by 3 subcutaneous injections of Epigen in Freunds incomplete adjuvant at 3 weekly intervals with the same protein. Antisera were collected from the rabbits and the IgG purified by Protein A affinity chromatography. The resultant polyclonal antibody recognized human and mouse TR1 in ELISAs and by Western blotting.

Immunohistochemical analysis of human normal and diseased tissue arrays (SuperBioChips Laboratories, Seoul, Korea) revealed that epithelial cells in skin, kidney, breast, small intestine and kidney all express moderate levels of TR1. The tissues that expressed significant levels of TR1 were the spleen, where TR1 was expressed in the cells that lined the venous structures of the red pulp, the glomerulus's of the kidney, the secretory goblet cells in the stomach and a transitional cell carcinoma in the bladder. It was clear from this and a previous study analyzing rat and human arteries, that the cells surrounding vessels, probably smooth muscle cells, express TR1.

Skin biopsies taken from psoriasis patients before and after treatment with delipidated and deglycolipidated M. vaccae (U.S. Pat. Nos. 5,985,287 and 6,328,978) were analyzed for TR1 expression. All of the differentiating keratinocytes expressed TR1, whereas the immature keratinocytes in the basal layer expressed little or no TR1.

The localization of TR1 in the tissues described above indicate that inhibitors of TR1 may be employed to:

(a) block aberrant smooth muscle cell growth in diseases and conditions such as atherosclerosis, cardiovascular diseases, leiomyosarcoma, fibroids and stent overgrowth;

(b) inhibit tumor growth including transitional cell carcinomas;

(c) inhibit diseases associated with the dysfunction of the secretory processes in the stomach such as ulceration; and

(d) inhibit aberrant epithelial cell growth associated with diseases such as psoriasis, Crohns disease and epithelial tumors.

### EXAMPLE 4

### Identification, Isolation and Characterization of DP3

A partial cDNA fragment, referred to as DP3, was identified by differential display RT-PCR (modified from Liang P and Pardee A B, Science 257:967-971, 1992) using mRNA from cultured rat dermal papilla and footpad fibroblast cells, isolated by standard cell biology techniques. This double stranded cDNA was labeled with [α³²P]-dCTP and used to identify a full length DP3 clone by screening 400,000 pfu's of an oligo dT-primed rat dermal papilla cDNA library. The determined full-length cDNA sequence for DP3 is provided in SEQ ID NO: 119, with the corresponding amino acid sequence being provided in SEQ ID NO: 197. Plaque lifts, hybridization and screening were performed using standard molecular biology techniques.

### EXAMPLE 5

### Isolation and Characterization of KS1

Analysis of RNA Transcripts by Northern Blotting

Northern analysis to determine the size and distribution of mRNA for muKS1 (SEQ ID NO: 263) was performed by probing murine tissue mRNA blots with a probe consisting of nucleotides 268-499 of muKS1, radioactively labeled with [α³²P]-dCTP. Prehybridization, hybridization, washing, and probe labeling were performed as described in Sambrook, et al., Ibid. mRNA for muKS1 was 1.6 kb in size and was observed to be most abundant in brain, lung, or any muscle, and heart. Expression could also be detected in lower intestine, skin, bone marrow, and kidney. No detectable signal was found in testis, spleen, liver, thymus, stomach.

Human Homologue of muKS1

MuKS1 (SEQ ID NO: 263) was used to search the EMBL database (Release 50, plus updates to June, 1998) to identify human EST homologues. The top three homologies were to the following ESTs: accession numbers AA643952, HS1301003 and AA865643. These showed 92.63% identity over 285 nucleotides, 93.64% over 283 nucleotides and 94.035% over 285 nucleotides, respectively. Frame shifts were identified in AA643952 and HS1301003 when translated. Combination of all three ESTs identified huKS1 (SEQ ID NO: 270) and translated polypeptide SEQ ID NO: 344. Alignment of muKS1 and huKS1 polypeptides indicated 95% identity over 96 amino acids.

Identification of KSCL009274 cDNA Sequence

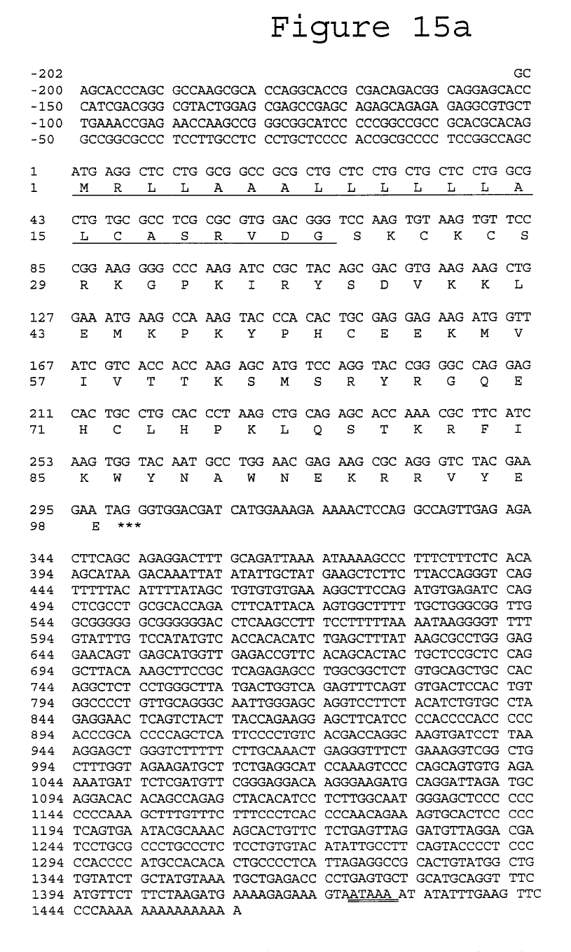

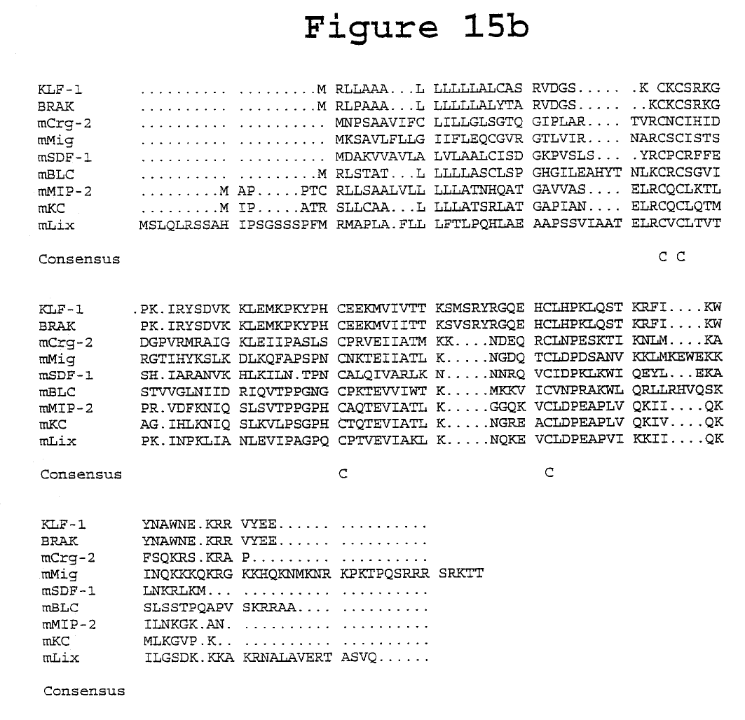

A directionally cloned cDNA library was constructed from immature murine keratinocytes and submitted for high-throughput sequencing. Sequence data from a clone designated KDCL009274 showed 35% identity over 72 amino acids with rat macrophage inflammatory protein-2B (MIP-2B) and 32% identity over 72 amino acids with its murine homologue. The insert of 1633bp (SEQ ID NO: 464; FIG. 15A) contained an open reading frame of 300bp with a 5′ untranslated region of 202bp and a 3′ untranslated region of 1161 bp. A poly-adenylation signal of AATAAA is present 19 base pairs upstream of the poly-A tail. The mature polypeptide (SEQ ID NO: 465) is 77 amino acids in length containing 4 conserved cysteines with no ELR motif. The putative signal peptide cleavage site beween GLY 22 and Ser 23 was predicted by the hydrophobicity profile. This putative chemokine was identical to KS1. The full length sequence was screened against the EMBL database using the BLAST program and showed some identity at the nucleotide level with human EST clones AA643952, AA865643, and HS1301003, respectively. A recently described human CXC chemokine, BRAK, has some identity with KS1 at the protein level. The alignment of KS1 (referred to in FIG. 15B as KLF-1), BRAK, and other murine α-chemokines is shown in FIG. 15B. The phylogenetic relationship between KS1 and other α-chemokine family members was determiend using the Phylip program. KS1 and BRAK demonstrate a high degree of divergence from the other α-chemokines, supporting the relatively low homology shown in the multiple alignment.

Bacterial Expression and Purification of muKS1 and huKS1

Polynucleotides 269-502 of muKS1 (SEQ ID NO: 271), encoding amino acids 23-99 of polypeptide muKS1 (SEQ ID NO: 345), and polynucleotides 55-288 of huKS1 (SEQ ID NO: 272), encoding amino acids 19-95 of polypeptide huKS1 (SEQ ID NO: 346), were cloned into the bacterial expression vector pET-16b (Novagen, Madison, Wis.), which contains a bacterial leader sequence and N-terminal 6×Histidine tag. These constructs were transformed into competent XL1-Blue E. coli as described in Sambrook et al., Ibid.

Starter cultures of recombinant BL 21 (DE3) E. coli (Novagen) containing SEQ ID NO: 271 (muKS1a) and SEQ ID NO: 272 (huKS1a) were grown in NZY broth containing 100 μg/ml ampicillin (Gibco-BRL Life Technologies) at 37° C. Cultures were spun down and used to inoculate 800 ml of NZY broth and 100 μg/ml ampicillin. Cultures were grown until the OD₅₉₅ of the cells was between 0.4 and 0.8. Bacterial expression was induced for 3 hours with 1 mM IPTG. Bacterial expression produced an induced band of approximately 15 kDa for muKS1a and huKS1a.

MuKS1a and huKS1a were expressed in insoluble inclusion bodies. In order to purify the polypeptides, bacterial cell pellets were re-suspended in lysis buffer (20 mM Tris-HCl pH 8.0, 10 mM βMercaptoethanol, 1 mM PMSF). To the lysed cells, 1% NP-40 was added and the mix incubated on ice for 10 minutes. Lysates were further disrupted by sonication on ice at 95 W for 4×15 seconds and then centrifuged for 10 minutes at 18,000 rpm to pellet the inclusion bodies.

The pellet containing the inclusion bodies was re-suspended in lysis buffer containing 0.5% w/v CHAPS and sonicated for 5-10 seconds. This mix was stored on ice for 1 hour, centrifuged at 14000 rpm for 15 minutes at 4° C. and the supernatant discarded. The pellet was once more re-suspended in lysis buffer containing 0.5% w/v CHAPS, sonicated, centrifuged, and the supernatant removed as before. The pellet was re-suspended in solubilizing buffer (6 M guanidine HCl, 0.5 M NaCl, 20 mM Tris-HCl pH 8.0), sonicated at 95W for 4×15 seconds and centrifuged for 10 minutes at 18000 rpm and 4° C. to remove debris. The supernatant was stored at 4° C. MuKS1a and huKS1a were purified by virtue of the N-terminal 6×histidine tag contained within the bacterial leader sequence, using a Nickel-Chelating sepharose column (Amersham Pharmacia, Uppsala, Sweden) and following the manufacturer's protocol. Proteins were purified twice over the column to reduce endotoxin contamination. In order to re-fold the proteins once purified, the protein solution was dialysed in a 4 M-2 M urea gradient in 20 mM tris-HCl pH 7.5+10% glycerol overnight at 4° C. The protein was then further dialysed 2× against 2 liters of 20 mM Tris-HCl pH 7.5+10% (w/v) glycerol. Preparations obtained were greater than 95% pure as determined by SDS-PAGE. Endotoxin contamination of purified proteins were determined using a limulus amebocyte lysate assay kit (BIO Whittaker, Walkersville, Md.). Endotoxin levels were <0.1 ng/μg of protein. Internal amino acid sequencing was performed on tryptic peptides of KS1.

An Fc fusion protein was produced by expression in HEK 293 T cells. 35 μg of KLF-1plGFc DNA to transfect 6×10⁶ cells per flask, 200 mls of Fc containing supernatant was produced. The Fc fusion protein was isolated by chromatography using an Affiprep protein A resin (0.3 ml column, Biorad). After loading, the column was washed with 15 mls of PBS, followed by a 5 ml wash of 50 mM Na citrate pH 5.0. The protein was then eluted with 6 column volumes of 50 mM Na citrate pH 2.5, collecting 0.3 ml fractions in tubes containing 60 μl of 2M Tris-HCl pH 8.0. Fractions were analyzed by SDS-PAGE.

Peptide Sequencing of muKS1 and huKS1

Bacterially expressed muKS1 and huKS1 were separated on polyacrylamide gels and induced bands of 15 kDa were identified. The predicted size of muKS1 is 9.4 kDa. To obtain the amino acid sequence of the 15 kDa bands, 20 μg recombinant muKS1 and huSK1 was resolved by SDS-PAGE and electroblotted onto Immobilon PVDF membrane (Millipore, Bedford, Mass.). Internal amino acid sequencing was performed on tryptic peptides of muKS1 and huKS1 by the Protein Sequencing Unit at the University of Auckland, New Zealand.

The determined amino acid sequences for muKS1 and huKS1 are given in SEQ ID NOS: 397 and 398, respectively. These amino acid sequences confirmed that the determined sequences are identical to those established on the basis of the cDNA sequences. The size discrepancy has previously been reported for other chemokines (Richmond A, Balentien E, Thomas H G, Flaggs G, Barton D E, Spiess J, Bordoni R, Francke U, Derynck R, “Molecular characterization and chromosomal mapping of melanoma growth stimulatory activity, a growth factor structurally related to beta-thromboglobulin,” EMBO J. 7:2025-2033, 1988; Liao F, Rabin R L, Yannelli J R, Koniaris L G, Vanguri P, Farber J M, “Human Nig chemokine: biochemical and functional characterization,” J. Exp. Med. 182:1301-1314, 1995). The isoelectric focusing point of these proteins was predicted to be 10.26 using DNASIS (HITACHI Software Engineering, San Francisco, Calif.). Recombinant Fc tagged KS1 expresssed and purified using protein A affinity column chromatography revealed a homogenous protein with a molecular mass of 42 kDa.

Oxidative Burst Assay

Oxidative burst assays were used to determine responding cell types. 1×10⁷ PBMC cells were resuspended in 5 ml HBSS, 20 mM HEPES, 0.5% BSA and incubated for 30 minutes at 37° C. with 5 μl 5 mM dichloro-dihydrofluorescein diacetate (H₂DCFDA, Molecular Probes, Eugene, Oreg.). 2×10⁵ H₂DCFDA-labeled cells were loaded in each well of a flat-bottomed 96 well plate. 10 μl of each agonist was added simultaneously into the well of the flat-bottomed plate to give final concentrations of 100 ng/ml (fMLP was used at 10 μM). The plate was then read on a Victor² 1420 multilabel counter (Wallac, Turku, Finland) with a 485 nm excitation wavelength and 535 nm emission wavelength. Relative fluorescence was measured at 5 minute intervals over 60 minutes.

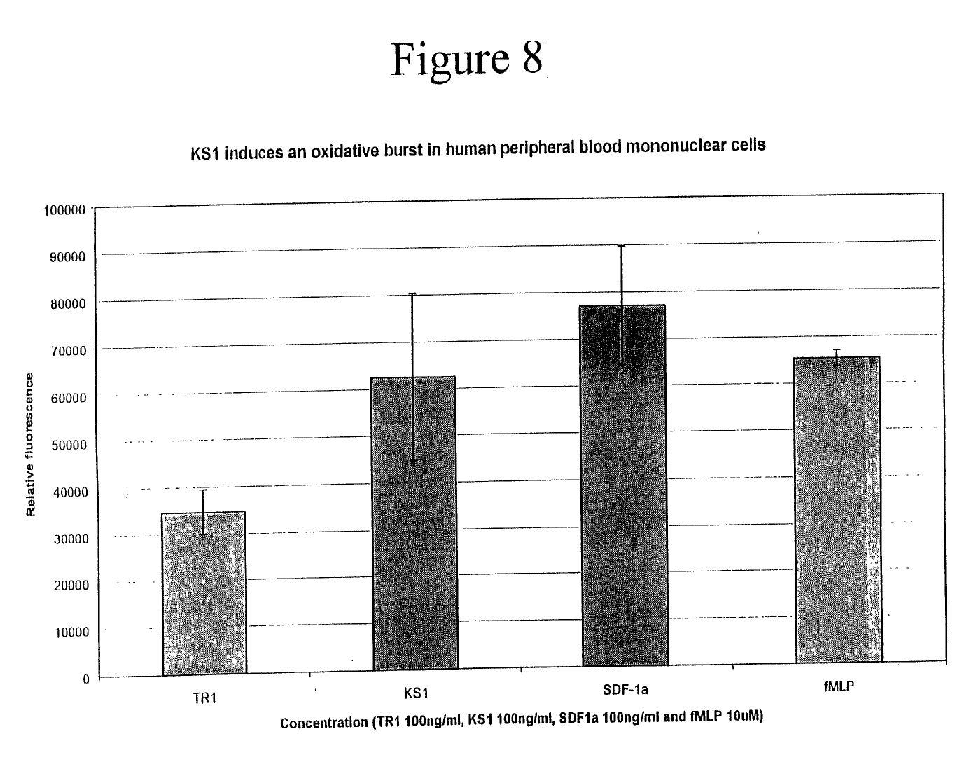

A pronounced respiratory burst was identified in PBMC with a 2.5 fold difference between control treated cells (TR1) and cells treated with 100 ng/ml muKS1 (FIG. 8). Human stromal derived factor-1α (SDF1α) (100 ng/ml) and 10 μM formyl-Met-Leu-Phe (fMLP) were used as positive controls.

Chemotaxis Assay

Cell migration in response to muKS1 was tested using a 48 well Boyden's chamber (Neuro Probe Inc., Cabin John, Md.) as described in the manufacturer's protocol. In brief, agonists were diluted in HBSS, 20 mM HEPES, 0.5% BSA and added to the bottom wells of the chemotactic chamber. THP-1 cells were re-suspended in the same buffer at 3×10⁵ cells per 50 μl. Top and bottom wells were separated by a PVP-free polycarbonate filter with a 5 μm pore size for monocytes or 3 μm pore size for lymphocytes. Cells were added to the top well and the chamber incubated for 2 hours for monocytes and 4 hours for lymphocytes in a 5% CO₂ humidified incubator at 37° C. After incubation, the filter was fixed and cells scraped from the upper surface. The filter was then stained with Diff-Quick (Dade International Inc., Miami, Fla.) and the number of migrating cells counted in five randomly selected high power fields. The results are expressed as a migration index (the number of test migrated cells divided by the number of control migrated cells).

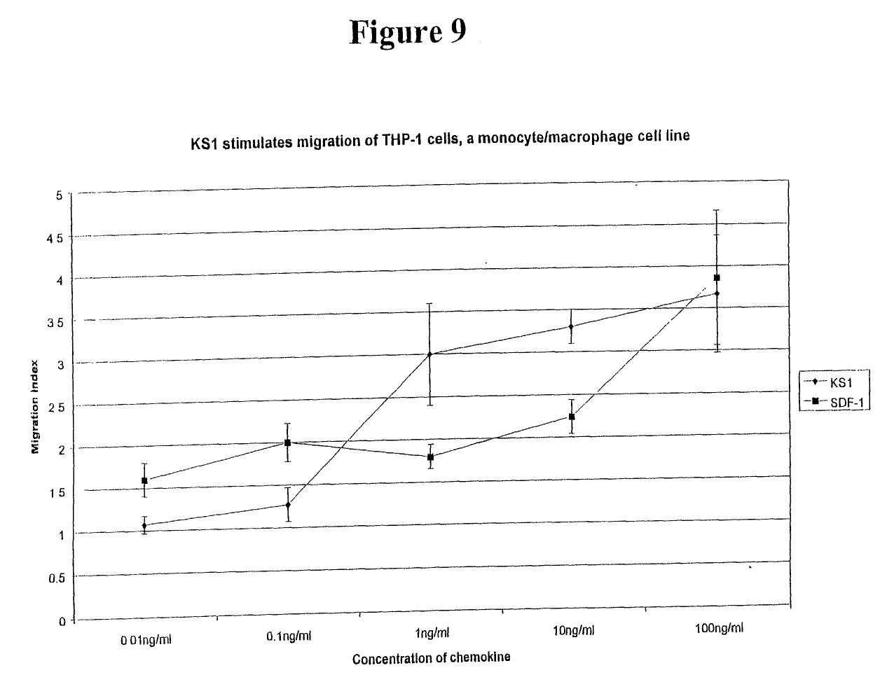

Using this assay, muKS1 was tested against T cells and THP-1 cells. MuKS1 induced a titrateable chemotactic effect on THP-1 cells from 0.01 ng/ml to 100 ng/ml (FIG. 9). Human SDF1α was used as a positive control and gave an equivalent migration. MuKS1 was also tested against IL-2 activated T cells. However, no migration was evidence for muKS1 even at high concentrations, whereas SDF-1α provided an obvious titrateable chemotactic stimulus. Therefore, muKS1 appears to be chemotactic for THP-1 cells but not for IL-2 activated T cells at the concentrations tested.

Flow Cytometric Binding Studies

Binding of KLF-1 to THP-1 and Jurkat cells was tested in the following manner. THP-1 or Jurkat cells (5×10⁶) were resuspended in 3 mls of wash buffer (2% FBS and 0.2% sodium azide in PBS) and pelleted at 4° C., 200×g for 5 minutes. Cells were then blocked with 0.5% mouse and goat sera for 30 minutes on ice. Cells were washed, pelleted, resuspended in 50 μl of KLF-1Fc at 10 μg/ml and incubated for 30 minutes on ice. After incubation, the cells were prepared as before and resuspended in 50 μl of goat anti-human IgG biotin (Southern Biotechnology Associates, AL) at 10 μg/ml and incuated for 30 minutes on ice. Finally, cells were washed, pelleted and resuspended in 50 μl of streptavidin-RPE (Southern Biotechnology Associates, AL) at 10 μg/ml and incuabated for a further 30 minutes on ice in the dark. Cells were washed and resuspended in 250 μl of wash buffer and stained with 1 μl of 10 μg/ml propidium iodide (Sigma) to exclude any dead cells. Purified Fc fragment (10 μg/ml) was used as a negative control in place of KLF-1Fc to determine non-specific binding. Ten thousand gated events were analyzed on log scale using PE filter arrangement with peak transmittance at 575 nm and bandwidth of 10 nm on an Elite cell sorter (Coulter Cytometry).

The respiratory burst and migration assays indicated that KS1 is active on monocytes and not T cells; therefore, the KS1 Fc fusion protein was tested in a binding study with THP-1 and Jurkat T cells. KS1 Fc showed a marked positive shift on THP-1 cells compared with the Fc fragment alone. In contrast, KS1 demonstrated no positive binding with Jurkat cells in an identical experiment.

Full Length Sequence of muKS1 Clone

The nucleotide sequence of muKS1 was extended by determining the base sequence of additional ESTs. Combination of all the ESTs identified the full-length muKS1 (SEQ ID NO: 370) and the corresponding translated polypeptide sequence in SEQ ID NO: 394.

Analysis of Human RNA Transcripts by Northern blotting

Northern blot analysis to determine the size and distribution of mRNA for the human homologue of muKS1 was performed by probing human tissue blots (Clontech, Palo Alto, Calif.) with a radioactively labeled probe consisting of nucleotides 1 to 288 of huKS1 (SEQ ID NO: 270). Prehybridization, hybridization, washing, and probe labeling were performed as described in Sambrook, et al., Ibid. mRNA for huKS1 was 1.6 kb in size and was observed to be most abundance in kidney, liver, colon, small intestine, and spleen. Expression could also be detected in pancreas, skeletal muscle, placenta, brain, heart, prostate, and thymus. No detectable signal was found in lung, ovary, and testis.

Analysis of Human RNA Transcripts in Tumor Tissue by Northern blotting

Northern blot analysis to determine distribution of huKS1 in cancer tissue was performed as described previously by probing tumor panel blots (Invitrogen, Carlsbad, Calif.). These blots make a direct comparison between normal and tumor tissue. mRNA was observed in normal uterine and cervical tissue but not in the respective tumor tissue. In contrast, expression was up-regulated in breast tumor and down-regulated in normal breast tissue. No detectable signal was found in either ovary or ovarian tumors.

Injection of Bacterially Recombinant muKS1 into C3H/HeJ Mice

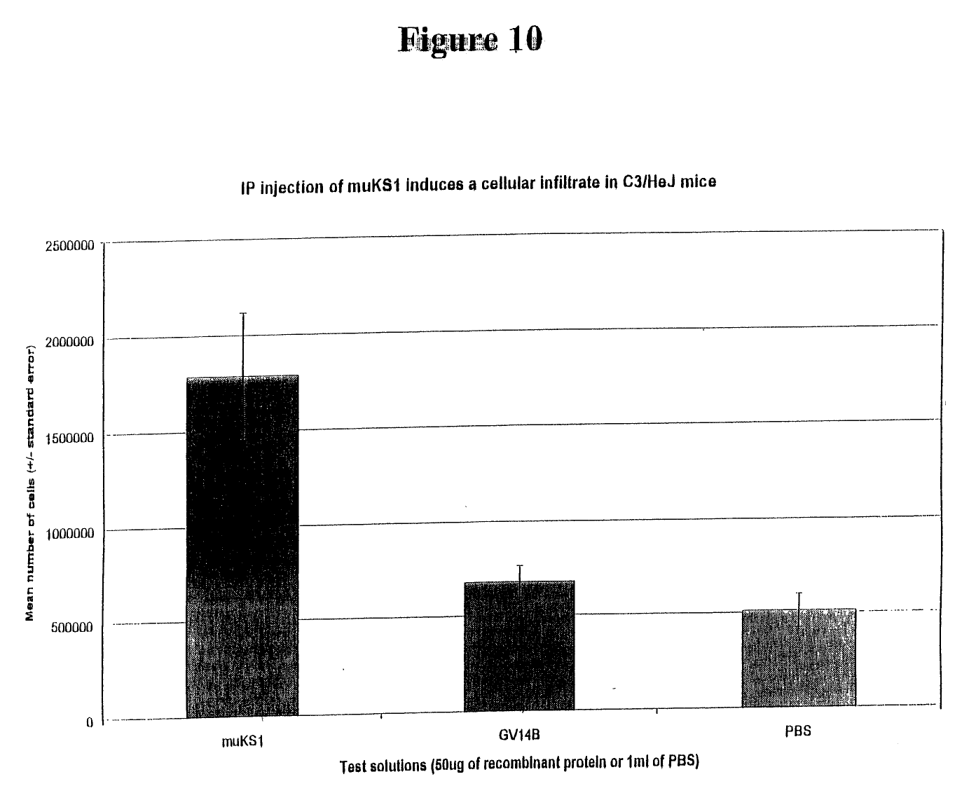

Eighteen C3H/HeJ mice were divided into 3 groups and injected intraperitoneally with muKS1, GV14B, or phosphate buffered saline (PBS). GV14B is a bacterially expressed recombinant protein used as a negative control. Group 1 mice were injected with 50 μg of muKS1 in 1 ml of PBS; Group 2 mice were injected with 50 μg of GV14B in 1 ml of PBS; and Group 3 mice with 1 ml of PBS. After 18 hours, the cells in the peritoneal cavity of the mice were isolated by intraperitoneal lavage with 2×4 ml washes with harvest solution (0.02% EDTA in PBS). Viable cells were counted from individual mice from each group. Mice injected with 50 μg of muKS1 had on average a 3-fold increase in cell numbers (FIG. 10).

20 μg of bacterial recombinant muKS1 was injected subcutaneously into the left hind foot of three C3H/HeJ mice. The same volume of PBS was injected into the same site on the right-hand side of the same animal. After 18 hours, mice were examined for inflammation. All mice showed a red swelling in the foot pad injected with bacterially recombinant KS1. From histology, sites injected with muKS1 had an inflammatory response of a mixed phenotype with mononuclear and polymorphonuclear cells present.

Injection of Bacterially Expressed muKS1a into Nude Mice

To determine whether T cells are required for the inflammatory response, the experiment was repeated using nude mice. Two nude mice were anaesthetized intraperitoneally with 75 μl of 1/10 dilution of Hypnorm (Janssen Pharmaceuticals, Buckinghamshire, England) in phosphate buffered saline. 20 ug of bacterially expressed muKS1a (SEQ ID NO: 345) was injected subcutaneously in the left hind foot, ear and left-hand side of the back. The same volume of phosphate buffered saline was injected in the same sites but on the right-hand side of the same animal. Mice were left for 18 hours and then examined for inflammation. Both mice showed a red swelling in the ear and foot sites injected with the bacterially expressed protein. No obvious inflammation could be identified in either back site. Mice were culled and biopsies taken from the ear, back and foot sites and fixed in 3.7% formol saline. Biopsies were embedded, sectioned and stained with Haemotoxylin and eosin. Sites injected with muKS1a had a marked increase in polymorphonuclear granulocytes, whereas sites injected with phosphate buffered saline had a low background infiltrate of polymorphonuclear granulocytes.

Discussion

Chemokines are a large superfamily of highly basic secreted proteins with a broad number of functions (Baggiolini, et al., Annu. Rev. Immunol., 15:675-705, 1997; Ward, et al., Immunity, 9:1-11, 1998; Horuk, Nature, 393:524-525, 1998). The polypeptide sequences of muKS1 and huKS1 have similarity to CXC chemokines, suggesting that this protein will act like other CXC chemokines. The in vivo data from nude mice supports this hypothesis. This chemokine-like protein may therefore be expected to stimulate leukocyte, epithelial, stromal, and neuronal cell migration; promote angiogenesis and vascular development; promote neuronal patterning, hemopoietic stem cell mobilization, keratinocyte and epithelial stem cell patterning and development, activation and proliferation of leukocytes; and promotion of migration in wound healing events. It has recently been shown that receptors to chemokines act as co-receptors for HIV-1 infection of CD4+cells (Cairns, et al., Nature Medicine, 4:563-568, 1998) and that high circulating levels of chemokines can render a degree of immunity to those exposed to the HIV virus (Zagury, et al., Proc. Natl. Acad. Sci. USA 95:3857-3861, 1998). This novel gene and its encoded protein may thus be usefully employed as regulators of epithelial, lymphoid, myeloid, stromal, and neuronal cells migration and cancers; as agents for the treatment of cancers, neuro-degenerative diseases, inflammatory autoimmune diseases such as psoriasis, asthma and Crohn's disease; for use in wound healing; and as agents for the prevention of HIV-1 binding and infection of leukocytes.

We have also shown that muKS1 promotes a quantifiable increase in cell numbers in the peritoneal cavity of C3H/HeJ mice injected with muKS1. Furthermore, we have shown that muKS1 induces an oxidative burst in human peripheral blood mononuclear cells and migration in the human monocyte leukemia cell line, THP-1, suggesting that monocyte/macrophages are one of the responsive cell types for KS1. In addition to this, we demonstrated that huKS1 was expressed at high levels in a number of non-lymphoid tissues, such as the colon and small intestine, and in breast tumors. It was also expressed in normal uterine and cervical tissue, but was completely down-regulated in their respective tumors. It has been shown that non-ELR chemokines have demonstrated angiostatic properties. IP-10 and Mig, two non-ELR chemokines, have previously been shown to be up-regulated during regression of tumors (Tannenbaum C S, Tubbs R, Armstrong D, Finke J H, Bukowski R M, Hamilton T A, “The CXC Chemokines IP-10 and Mig are necessary for IL-12-mediated regression of the mouse RENCA tumor,” J. Immunol. 161: 927-932, 1998), with levels of expression inversely correlating with tumor size (Kanegane C, Sgadari C, Kanegane H, Teruya-Feldstine J, Yao O, Gupta G, Farber J M, Liao F, Liu L, Tosato G, “Contribution of the CXC Chemokines IP-10 and Mig to the antitumor effects of IL-12,” J. Leuko. Biol. 64: 384-392, 1998). Furthermore, neutralizing antibodies to IP-10 and Mig would reduce the anti-tumor effect, indicating the contribution these molecules make to the anti-tumor effects. Therefore, it is expected that in the case of cervical and uterine tumors, KS1 would have similar properties.

The data demonstrates that KS1 is involved in cell migration showing that one of the responsive cell types is monocyte/macrophage. The human expression data in conjunction with the in vitro and in vivo biology demonstrates that this molecule may be a useful regulator in cell migration, and as an agent for the treatment of inflammatory diseases, such as Crohn's disease, ulcerative colitis, and rheumatoid arthritis; and cancers, such as cervical adenocarcinoma, uterine leiomyoma, and breast invasive ductal carcinoma.

### EXAMPLE 6

### Characterization of KS2

KS2 contains a transmembrane domain and may function as either a membrane-bound ligand or a receptor. Northern analysis indicated that the mRNA for KS2 was expressed in the mouse keratinocyte cell line, Pam212, consistent with the cDNA being identified in mouse keratinocytes.

Mammalian Expression

To express KS2, the extracellular domain was fused to the amino terminus of the constant domain of immunoglobulin G (Fc) that had a C-terminal 6×Histidine tag. This was performed by cloning polynucleotides 20-664 of KS2 (SEQ ID NO: 273), encoding amino acids 1-215 of polypeptide KS2 (SEQ ID NO: 347), into the mammalian expression vector pcDNA3 (Invitrogen, NV Leek, Netherlands), to the amino terminus of the constant domain of immunoglobulin G (Fc) that had a C-terminal 6×Histidine tag. This construct was transformed into competent XL1-Blue E. coli as described in Sambrook et al., Ibid. The Fc fusion construct of KS2a was expressed by transfecting Cos-1 cells in 5×T175 flasks with 180 μg of KS1a using DEAE-dextran. The supernatant was harvested after seven days and passed over a Ni-NTA column. Bound KS2a was eluted from the column and dialyzed against PBS.

The ability of the Fc fusion polypeptide of KS2a to inhibit the IL-2 induced growth of concanavalin A stimulated murine splenocytes was determined as follows. A single cell suspension was prepared from the spleens of BALB/c mice and washed into DMEM (GIBCO-BRL) supplemented with 2 mM L-glutamine, 1 mM sodium pyruvate, 0.77 mM L-asparagine, 0.2 mM L-arganine, 160 mM penicillin G, 70 mM dihydrostreptomycin sulfate, 5×10⁻² mM beta mercaptoethanol and 5% FCS (cDMEM). Splenocytes (4×10⁶/ml) were stimulated with 2 μg/ml concanavalin A for 24 hrs at 37° C. in 10% CO₂. The cells were harvested from the culture, washed 3 times in cDMEM and resuspended in cDMEM supplemented with 10 ng/ml rhuIL-2 at 1×10⁵ cells/ml. The assay was performed in 96 well round bottomed plates in 0.2 ml cDMEM. The Fc fusion polypeptide of KS2a, PBS, LPS and BSA were titrated into the plates and 1×10⁴ activated T cells (0.1 ml) were added to each well. The plates were incubated for 2 days in an atmosphere containing 10% CO₂ at 37° C. The degree of proliferation was determined by pulsing the cells with 0.25 uCi/ml tritiated thymidine for the final 4 hrs of culture after which the cells were harvested onto glass fiber filtermats and the degree of thymidine incorporation determined by standard liquid scintillation techniques. As shown in FIG. 6, the Fc fusion polypeptide of KS2a was found to inhibit the IL-2 induced growth of concanavalin A stimulated murine splenocytes, whereas the negative controls PBS, BSA and LPS did not.

This data demonstrates that KS2 is expressed in skin keratinocytes and inhibits the growth of cytokine induced splenocytes. This indicates a role for KS2 in the regulation of skin inflammation and malignancy.

### EXAMPLE 7

### Characterization of KS3

KS3 encodes a polypeptide of 40 amino acids (SEQ ID NO: 129). KS3 contains a signal sequence of 23 amino acids that would result in a mature polypeptide of 17 amino acids (SEQ ID NO: 348; referred to as KS3a).

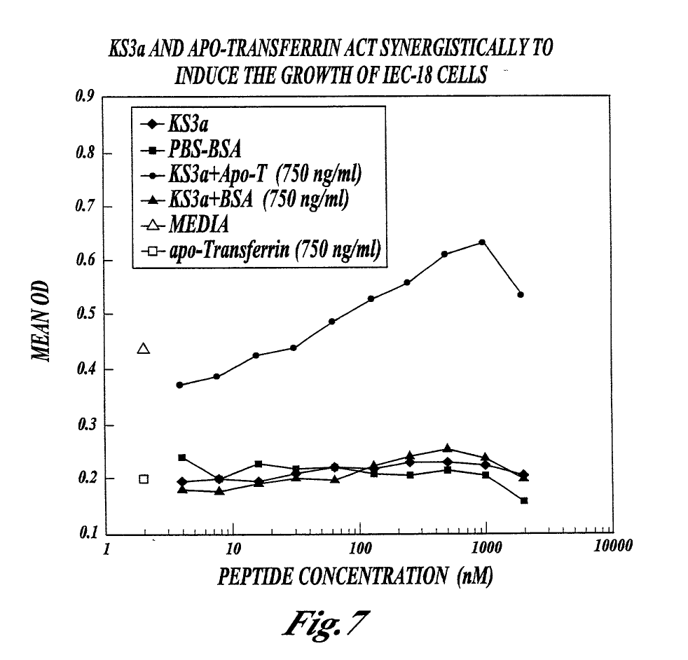

KS3a was prepared synthetically (Chiron Technologies, Victoria, Australia) and observed to enhance transferrin-induced growth of the rat intestinal epithelial cells IEC-18 cells. The assay was performed in 96 well flat-bottomed plates in 0.1 ml DMEM (GIBCO-BRL Life Technologies) supplemented with 0.2% FCS. KS3a (SEQ ID NO: 348), apo-Transferrin, media and PBS-BSA were titrated either alone, with 750 ng/ml Apo-transferrin or with 750 ng/ml BSA, into the plates and 1×10³ IEC-18 cells were added to each well. The plates were incubated for 5 days at 37° C. in an atmosphere containing 10% CO₂. The degree of cell growth was determined by MTT dye reduction as described previously (J. Imm. Meth. 93:157-165, 1986). As shown in FIG. 7, KS3a plus Apo-transferrin was found to enhance transferrin-induced growth of IEC-18 cells, whereas KS3a alone or PBS-BSA did not, indicating that KS3a and Apo-transferrin act synergistically to induce the growth of IEC-18 cells.

This data indicates that KS3 is epithelial derived and stimulates the growth of epithelial cells of the intestine. This suggests a role for KS3 in wound healing, protection from radiation- or drug-induced intestinal disease, and integrity of the epithelium of the intestine.

SEQ ID NOS: 1-725 are set out in the attached Sequence Listing. The codes for polynucleotide and polypeptide sequences used in the attached Sequence Listing confirm to WIPO Standard ST.25 (1988), Appendix 2.

All references cited herein, including patent references and non-patent references, are hereby incorporated by reference in their entireties.

Although the present invention has been described in terms of specific embodiments, changes and modifications can be carried out without departing from the scope of the invention which is intended to be limited only by the scope of the appended claims.

We claim:

1. An isolated polypeptide comprising a sequence selected from the group consisting of: SEQ ID NO: 120-197, 275-348, 373-398, 406-409, 413-415, 417, 456-463, 465, 488-509, 512, 513 and 624-725.

2. An isolated polypeptide comprising a sequence selected from the group consisting of:
(a) sequences having at least 75% identity to a sequence provided in SEQ ID NO: 120-197, 275-348, 373-398, 406-409, 413-415, 417, 456-463, 465, 488-509, 512, 513 and 624-725;
(b) sequences having at least 90% identity to a sequence provided in SEQ ID NO: 120-197, 275-348, 373-398, 406-409, 413-415, 417, 456-463, 465, 488-509, 512, 513 and 624-725; and
(c) sequences having at least 95% identity to a sequence provided in SEQ ID NO: 120-197, 275-348, 373-398, 406-409, 413-415, 417, 456-463, 465, 488-509, 512, 513 and 624-725,
wherein the polypeptide possesses at least one functional property that is substantially the same as a functional property of a sequence of SEQ ID NO: 120-197, 275-348, 373-398, 406-409, 413-415, 417, 456-463, 465, 488-509, 512, 513 and 624-725.

3. An isolated polynucleotide that encodes a polypeptide according to any one of claims 1 and 2.

4. An isolated polynucleotide of claim 3, wherein the polynucleotide comprises a sequence selected from the group consisting of: sequences provided in SEQ ID NO: 1-119, 198-276, 349-372, 399-405, 410-412, 416, 418-455, 464, 466-487, 510, 511 and 514-623.

5. An isolated polynucleotide comprising a sequence selected from the group consisting of:
(a) complements of a sequence provided in SEQ ID NO: 1-119, 198-276, 349-372, 399-405, 410-412, 416, 418-455, 464, 466-487, 510, 511 and 514-623;
(b) reverse complements of a sequence provided in SEQ ID NO: 1-119, 198-276, 349-372, 399-405, 410-412, 416, 418-455, 464, 466-487, 510, 511 and 514-623;
(c) reverse sequences of a sequence provided in SEQ ID NO: 1-119, 198-276, 349-372, 399-405, 410-412, 416, 418-455, 464, 466-487, 510, 511 and 514-623;
(d) sequences having at least 75% identity to a sequence provided in SEQ ID NO: 1-119, 198-276, 349-372, 399-405, 410-412, 416, 418-455, 464, 466-487, 510, 511 and 514-623;
(e) sequences having at least 90% identity to a sequence provided in SEQ ID NO: 1-119, 198-276, 349-372, 399-405, 410-412, 416, 418-455, 464, 466-487, 510, 511 and 514-623; and
(f) sequences having at least 95% identity to a sequence of SEQ ID NO: 1-119, 198-276, 349-372, 399-405, 410-412, 416, 418-455, 464, 466-487, 510, 511 and 514-623.

6. An isolated polynucleotide comprising a sequence selected from the group consisting of:
(a) sequences that are a 200-mer of an isolated polynucleotide according to any one of claims 3, 4 and 5;
(b) sequences that are a 100-mer of an isolated polynucleotide according to any one of claims 3, 4 and 5; and
(c) sequences that are a 40-mer of an isolated polynucleotide according to any one of claims 3, 4 and 5.

7. An expression vector comprising an isolated polynucleotide according to any one of claims 3-6.

8. A host cell transformed with an expression vector according to claim 7.

9. An isolated polypeptide comprising at least a functional portion of an amino acid sequence selected from the group consisting of sequences provided in SEQ ID NO: 120-197, 275-348, 373-398, 406-409, 413-415, 417, 456-463, 465, 488-509, 512, 513 and 624-725.

10. A fusion protein comprising at least one polypeptide according to any one of claims 1, 2 and 9.

11. An isolated antibody, or antigen-binding fragment thereof, that specifically binds to a polypeptide of any one of claims 1 and 2.

12. A composition comprising an isolated polypeptide according to any one of claims 1, 2 and 9, and at least one component selected from the group consisting of: physiologically acceptable carriers and immunostimulants.

13. A composition comprising an isolated polynucleotide according to any one of claims 3-6 and at least one component selected from the group consisting of: physiologically acceptable carriers and immunostimulants.

14. A composition comprising a fusion protein according to claim 10 and at least one component selected from the group consisting of: physiologically acceptable carriers and immunostimulants.

15. A method for treating a disorder in a patient, comprising administering to the patient a composition comprising of any one of claims 12-14.

16. The method of claim 15, wherein the disorder is selected from the group consisting of: inflammatory disorders; cancer; and neurological disorders.

17. A method for stimulating keratinocyte growth and motility in a patient, comprising administering to the patient a composition of any one of claims 12-14.

18. A method for inhibiting the growth of cancer cells in a patient, comprising administering to the patient a composition of any one of claims 12-14.

19. A method for modulating angiogenesis in a patient, comprising administering to the patient a composition of any one of claims 12-14.

20. A method for inhibiting angiogenesis and vascularization of tumors in a patient, comprising administering to a patient a composition of any one of claims 12-14.

21. A method for modulating skin inflammation in a patient, comprising administering to the patient a composition of any one of claims 12-14.

22. A method for stimulating the growth of epithelial cells in a patient, comprising administering to the patient a composition of any one of claims 12-14.

23. A method for inhibiting the binding of HIV-1 to leukocytes in a patient, comprising administering to the patient a composition of any one of claims 12-14.

24. A method for treating a disorder, comprising reducing the amount or activity of a polypeptide of any one of claims 1, 2 and 9.

25. The method of claim 24, comprising administering an antibody of claim 11.

26. The method of claim 24, comprising administering an anti-sense oligonucleotide that binds specifically to a polynucleotide of any one of claims 3-6.

27. The method of claim 24, comprising administering a small interfering RNA molecule that corresponds to a polynucleotide of any one of claims 3-6.

28. The method of claim 24, wherein the disorder is characterized by tumor growth, aberrant epithelial cell growth or aberrant smooth muscle growth.

29. The method of claim 24, wherein the disorder is selected from the group consisting of: atherosclerosis, cardiovascular disease, leiomyosarcoma, fibroids, psoriasis, Crohns disease and epithelial cancers.
